(19) United States
(12) Patent Application Publication
(10) Pub. No.: US 2009/0018542 A1
(43) Pub. Date: Jan. 15, 2009

(54) FRACTURE FIXATION DEVICES, SYSTEMS AND METHODS INCORPORATING A MEMBRANE

(75) Inventors: Heber Saravia, Santa Rosa, CA (US); Brian A. Hauck, Windsor, CA (US); Charles L. Nelson, Santa Rosa, CA (US)

Correspondence Address: SHAY GLENN LLP, 2755 CAMPUS DRIVE, SUITE 210, SAN MATEO, CA, 94403, US

(73) Assignee: Sonoma Orthopedic Products,Inc., Santa Rosa, CA (US)

(21) Appl. No.: 12/172,146

(22) Filed: Jul. 11, 2008

Related U.S. Application Data
(60) Provisional application No. 60/949,071, filed on Jul. 11, 2007.

(51) Int. Cl. A61B 17/56

(52) U.S. Cl. 606/63; 606/60; 606/62; 606/64

(57) ABSTRACT

A bone fixation device having an elongate body, an actuateable gripper disposed on the elongated body, an actuator operably connected to the gripper to deploy the gripper from a retracted configuration to an expanded configuration, and a membrane surrounding at least a portion of the elongate body or the gripper is disclosed. Also disclosed are systems, surgical kits and methods of using a bone fixation device with a membrane cover.

### CROSS-REFERENCE TO RELATED APPLICATIONS

This application claims the benefit under 35 U.S.C. § 119 of U.S. provisional application Ser. No. 60/949,071, entitled “FRACTURE FIXATION DEVICE, TOOLS AND METHODS”, filed Jul. 11, 2007, the disclosure of which is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference

### BACKGROUND OF THE INVENTION

The present invention relates to methods and systems for providing reinforcement of bones. More specifically, the present invention relates to methods and systems for providing reconstructive surgical procedures and devices for reconstruction and reinforcement bones, including diseased, osteoporotic and fractured bones.

Bone fractures are a common medical condition both in the young and old segments of the population. However, with an increasingly aging population, osteoporosis has become more of a significant medical concern in part due to the risk of osteoporotic fractures. Osteoporosis and osteoarthritis are among the most common conditions to affect the musculoskeletal system, as well as frequent causes of locomotor pain and disability. Osteoporosis can occur in both human and animal subjects (e.g. horses). Osteoporosis (OP) and osteoarthritis (OA) occur in a substantial portion of the human population over the age of fifty. The National Osteoporosis Foundation estimates that as many as 44 million Americans are affected by osteoporosis and low bone mass, leading to fractures in more than 300,000 people over the age of 65. In 1997 the estimated cost for osteoporosis related fractures was $13 billion. That figure increased to $17 billion in 2002 and is projected to increase to $210-240 billion by 2040. Currently it is expected that one in two women, and one in four men, over the age of 50 will suffer an osteoporosis-related fracture. Osteoporosis is the most important underlying cause of fracture in the elderly. Also, sports and work-related accidents account for a significant number of bone fractures seen in emergency rooms among all age groups.

One current treatment of bone fractures includes surgically resetting the fractured bone. After the surgical procedure, the fractured area of the body (i.e., where the fractured bone is located) is often placed in an external cast for an extended period of time to ensure that the fractured bone heals properly. This can take several months for the bone to heal and for the patient to remove the cast before resuming normal activities.

In some instances, an intramedullary (IM) rod or nail is used to align and stabilize the fracture. In that instance, a metal rod is placed inside a canal of a bone and fixed in place, typically at both ends. See, for example, Fixion™ IM(Nail), www.disc-o-tech.com. This approach requires incision, access to the canal, and placement of the IM nail. The nail can be subsequently removed or left in place. A conventional IM nail procedure requires a similar, but possibly larger, opening to the space, a long metallic nail being placed across the fracture, and either subsequent removal, and or when the nail is not removed, a long term implant of the IM nail. The outer diameter of the IM nail must be selected for the minimum inside diameter of the space. Therefore, portions of the IM nail may not be in contact with the canal. Further, micro-motion between the bone and the IM nail may cause pain or necrosis of the bone. In still other cases, infection can occur. The IM nail may be removed after the fracture has healed. This requires a subsequent surgery with all of the complications and risks of a later intrusive procedure.

External fixation is another technique employed to repair fractures. In this approach, a rod may traverse the fracture site outside of the epidermis. The rod is attached to the bone with trans-dermal screws. If external fixation is used, the patient will have multiple incisions, screws, and trans-dermal infection paths. Furthermore, the external fixation is cosmetically intrusive, bulky, and prone to painful inadvertent manipulation by environmental conditions such as, for example, bumping into objects and laying on the device.

Other concepts relating to bone repair are disclosed in, for example, U.S. Pat. No. 5,108,404 to Scholten for Surgical Protocol for Fixation of Bone Using Inflatable Device; U.S. Pat. No. 4,453,539 to Raftopoulos et al. for Expandable Intramedullary Nail for the Fixation of Bone Fractures; U.S. Pat. No. 4,854,312 to Raftopolous for Expanding Nail; U.S. Pat. No. 4,932,969 to Frey et al. for Joint Endoprosthesis; U.S. Pat. No. 5,571,189 to Kuslich for Expandable Fabric Implant for Stabilizing the Spinal Motion Segment; U.S. Pat. No. 4,522,200 to Stednitz for Adjustable Rod; U.S. Pat. No. 4,204,531 to Aginsky for Nail with Expanding Mechanism; U.S. Pat. No. 5,480,400 to Berger for Method and Device for Internal Fixation of Bone Fractures; U.S. Pat. No. 5,102,413 to Poddar for Inflatable Bone Fixation Device; U.S. Pat. No. 5,303,718 to Krajicek for Method and Device for the Osteosynthesis of Bones; U.S. Pat. No. 6,358,283 to Hogfors et al. for Implantable Device for Lengthening and Correcting Malpositions of Skeletal Bones; U.S. Pat. No. 6,127,597 to Beyar et al. for Systems for Percutaneous Bone and Spinal Stabilization, Fixation and Repair; U.S. Pat. No. 6,527,775 to Warburton for Interlocking Fixation Device for the Distal Radius; U.S. Patent Publication US2006/0084998 A1 to Levy et al. for Expandable Orthopedic Device; and PCT Publication WO 2005/112804 A1 to Myers Surgical Solutions, LLC for Fracture Fixation and Site Stabilization System. Other fracture fixation devices, and tools for deploying fracture fixation devices, have been described in: U.S. Patent Appl. Publ. No. 2006/0254950; U.S. Ser. No. 60/867,011 (filed Nov. 22, 2006); U.S. Ser. No. 60/866,976 (filed Nov. 22, 2006); and U.S. Ser. No. 60/866,920 (filed Nov. 22, 2006).

In view of the foregoing, it would be desirable to have a device, system and method for providing effective and minimally invasive bone reinforcement and fracture fixation to treat fractured or diseased bones.

### SUMMARY OF THE INVENTION

Fracture fixation devices, and tools for deploying fracture fixation devices, have been described. See, e.g., U.S. Patent Appl. Publ. No. 2006/0254950; U.S. Ser. No. 60/867,011 (filed Nov. 22, 2006); U.S. Ser. No. 60/866,976 (filed Nov. 22, 2006); and U.S. Ser. No. 60/866,920 (filed Nov. 22, 2006).

The fracture fixation device of the invention is adapted to be inserted through an opening of a fractured bone, such as the radius (e.g., through a bony protuberance on a distal or proximal end or through the midshaft) into the intramedullary canal of the bone. In some embodiments, the fixation device has two main components, one configured component for being disposed on the side of the fracture closest to the opening and one component configured for being disposed on the other side of the fracture from the opening so that the fixation device traverses the fracture.

The device components cooperate to align, fix and/or reduce the fracture so as to promote healing. The device may be removed from the bone after insertion (e.g., after the fracture has healed or for other reasons), or it may be left in the bone for an extended period of time or permanently.

In some embodiments, the fracture fixation device has one or more actuatable anchors or grippers on its proximal and/or distal ends. These anchors may be used to hold the fixation device to the bone while the bone heals.

In some embodiments, to aid in insertion into the intramedullary canal, at least one component of the fracture fixation device has a substantially flexible state and a substantially rigid state. Once in place, deployment of the device also causes the components to change from the flexible state to a rigid state to aid in proper fixation of the fracture. At least one of the components may be substantially rigid or semi-flexible. At least one component may provide a bone screw attachment site for the fixation device.

In some embodiments, a bone fixation device is provided which includes an elongate body, an actuateable gripper disposed on the elongated body, an actuator operably connected to the gripper to deploy the gripper from a retracted configuration to an expanded configuration, and a membrane surrounding at least a portion of the elongate body or the gripper.

In some embodiments, a surgical kit is provided which includes a bone fixation device having an elongate body, an actuateable gripper disposed on the elongated body, and an actuator operably connected to the gripper to deploy the gripper from a retracted configuration to an expanded configuration, and a membrane configured to surround at least a portion of the bone fixation device.

In some embodiments, a method of repairing a fracture of a bone is provided. One such method includes covering at least a portion of a bone fixation device with a flexible membrane, inserting the device and the membrane into an intramedullary space of a bone to place a first portion of the device on one side of a fracture and a second portion of the device on another side of the fracture; and operating an actuator to deploy at least one gripper of the device to engage an inner surface of the intramedullary space to anchor the fixation device to the bone.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 shows a fracture fixation device according to one embodiment of this invention and two possible insertion paths: one near the bone end and another at midshaft.

FIGS. 2 and 3 show the fracture fixation device of FIG. 1 in place within fractured bones.

FIG. 4 shows an embodiment of a fracture fixation device with a curved hub at one end and a straight hub at another end.

FIG. 5 shows another embodiment of a fracture fixation device inserted into an intramedullary space of a fractured bone.

FIGS. 6 and 7 show yet another embodiment of a fracture fixation device being inserted into a fractured bone.

FIG. 8 shows the device of FIG. 6 in an undeployed configuration.

FIG. 9 is a cross-sectional view of the device of FIG. 8.

FIG. 10 shows the device of FIG. 6 in a deployed configuration.

FIG. 11 is a cross-sectional view of the device of FIG. 10.

FIG. 12 shows the deployed device of FIGS. 10 and 11 within a fractured bone.

FIGS. 13-18 show details of one embodiment of an actuatable gripper for use with a fracture fixation device.

FIGS. 19-21 show yet another embodiment of a fracture fixation device according to the invention.

FIG. 22 shows a portion of fracture fixation device of FIGS. 19-21 in a deployed configuration.

FIGS. 23-25 show the fracture fixation device of FIGS. 19-22 deployed within a bone.

FIGS. 26-31 show details of a gripper for use with a fracture fixation device.

FIGS. 32 and 33 show yet another embodiment of a fracture fixation device according to the invention.

FIGS. 34-39 show a deployment tool for use with a fracture fixation device of this invention.

FIGS. 40-41 show another embodiment of a deployment tool for use with a fracture fixation device of this invention.

FIGS. 42-43 show the interaction between a flexible screw driver and the actuator of a fixation device.

FIGS. 44-48 show another embodiment of a gripper for use with a fracture fixation device.

FIGS. 49-50 show another embodiment of a fracture fixation device similar to the device shown in FIGS. 19-25 using a distal gripper similar to that shown in FIGS. 44-48 but using an alternative actuator/locking mechanism.

FIGS. 51-52 show yet another embodiment of a fracture fixation device similar to the device shown in FIGS. 49-50 but using another alternative actuator and an alternative flexible body.

FIGS. 53-59 show alternative designs for the flexible body of a fracture fixation device according to this invention.

FIGS. 60 and 61 show an alternative embodiment of a fracture fixation device according to this invention.

### DETAILED DESCRIPTION OF THE INVENTION

By way of background and to provide context for the invention, it may be useful to understand that bone is often described as a specialized connective tissue that serves three major functions anatomically. First, bone provides a mechanical function by providing structure and muscular attachment for movement. Second, bone provides a metabolic function by providing a reserve for calcium and phosphate. Finally, bone provides a protective function by enclosing bone marrow and vital organs. Bones can be categorized as long bones (e.g. radius, femur, tibia and humerus) and flat bones (e.g. skull, scapula and mandible). Each bone type has a different embryological template. Further each bone type contains cortical and trabecular bone in varying proportions. The devices of this invention can be adapted for use in any of the bones of the body as will be appreciated by those skilled in the art.

Cortical bone (compact) forms the shaft, or diaphysis, of long bones and the outer shell of flat bones. The cortical bone provides the main mechanical and protective function. The trabecular bone (cancellous) is found at the end of the long bones, or the epiphysis, and inside the cortex of flat bones. The trabecular bone consists of a network of interconnecting trabecular plates and rods and is the major site of bone remodeling and resorption for mineral homeostasis. During development, the zone of growth between the epiphysis and diaphysis is the metaphysis. Finally, woven bone, which lacks the organized structure of cortical or cancellous bone, is the first bone laid down during fracture repair. Once a bone is fractured, the bone segments are positioned in proximity to each other in a manner that enables woven bone to be laid down on the surface of the fracture. This description of anatomy and physiology is provided in order to facilitate an understanding of the invention. Persons of skill in the art will also appreciate that the scope and nature of the invention is not limited by the anatomy discussion provided. Further, it will be appreciated there can be variations in anatomical characteristics of an individual patient, as a result of a variety of factors, which are not described herein. Further, it will be appreciated there can be variations in anatomical characteristics between bones which are not described herein

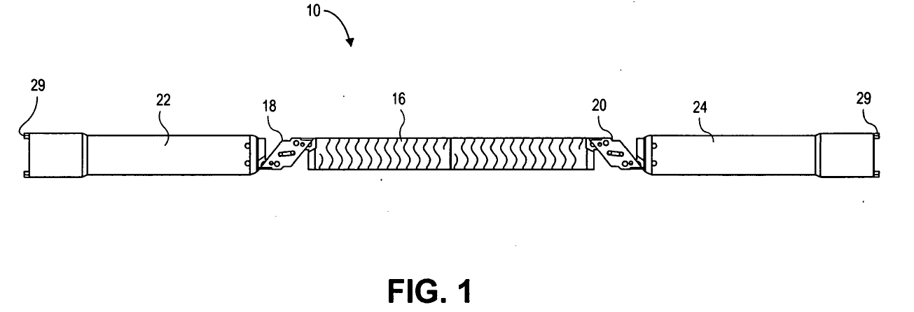

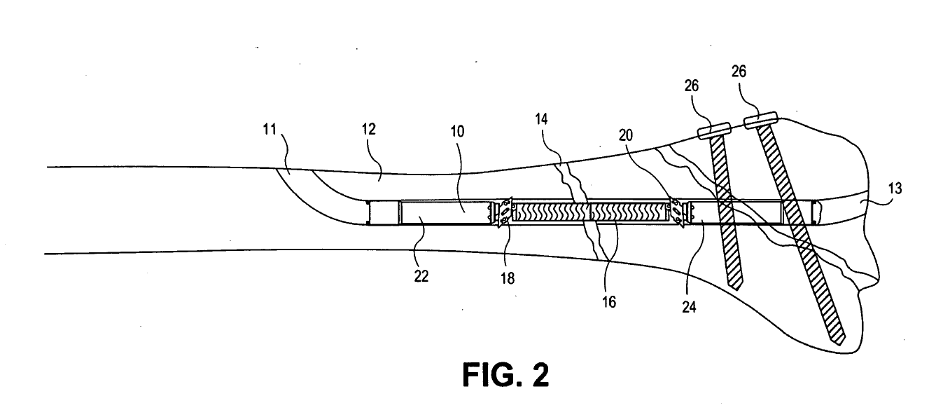

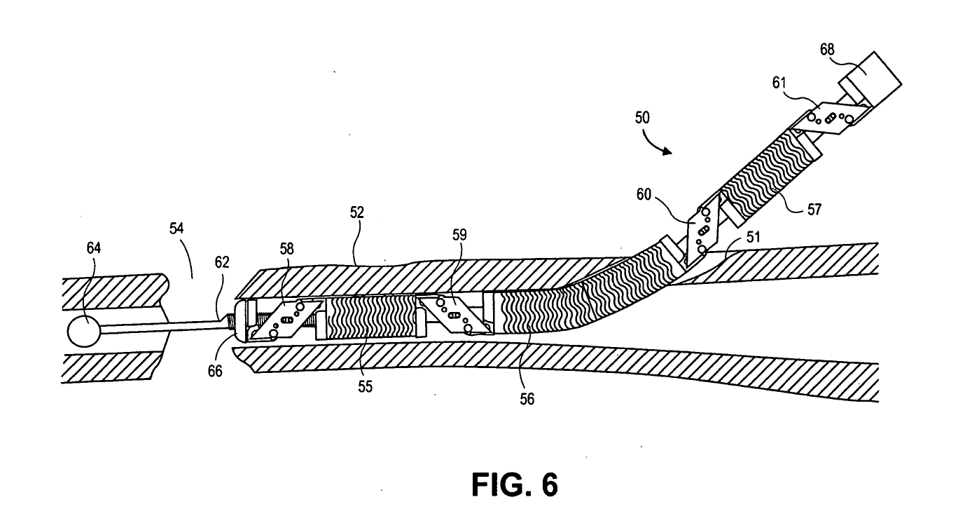

FIG. 1 shows a fracture fixation device 10 according to one embodiment of the invention. FIG. 2 shows device 10 in place within an intramedullary space of a fracture bone 12 and spanning a fracture site 14 after having been inserted an opening 11 or 13 formed in the bone 12. Device 10 has a flexible-to-rigid component 16 that may be compressed by an actuator (such as the actuator 62 shown in FIG. 6) from its flexible state to be made substantially rigid. Device 10 also has two actuatable grippers 18 and 20 and two hubs 22 and 24. In the configuration shown in FIG. 1, flexible-to-rigid component 16 is in its flexible state, and grippers 18 and 20 are in their unactuated state. In this embodiment, component 16 is formed as a unitary spirally wound element with wavy intersections between the turns. In the flexible state, there is a slight gap between the turns to permit the component 16 to bend. When compressed by an actuator, component 16 is foreshortened to bring the wavy spiral turns into compressive contact, thereby making component 16 rigid. The waves of the spiral also interact to permit the device to bend and to transmit torque, even in the device's flexible state.

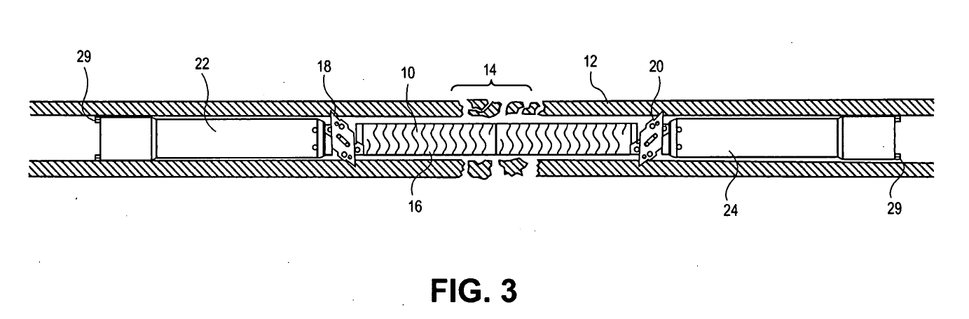

In FIGS. 2 and 3, component 16 has been actuated to its rigid state, and grippers 18 and 20 have been actuated to grip the interior of the bone. Grippers 18 and 20 in conjunction with flexible-to-rigid member 16 provide fixation to stabilize bone fragments. Optionally, one or more bone screws 26 may be inserted through the bone or fragments thereof into either or both hubs 22 and 24 to stabilize the device within the bone and to fix any bone fragments to the device as shown in FIG. 2. The bone screws can be inserted at any point along the hub(s) along any orientation desired. Attachment features 29 may be provided on the hubs to permit attachment of the fixation device to a deployment tool. FIG. 3 shows device 10 in use to repair a fracture having multiple bone fragments at the fracture site 14.

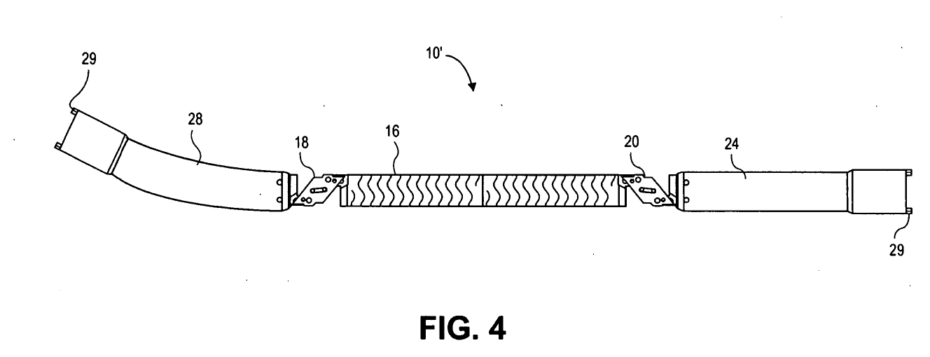

FIG. 4 shows an embodiment of a fracture fixation device 10′ similar to device 10 of FIGS. 3 and 1. In this embodiment, the straight hub at one end of the device has been replaced with a curved hub 28. The curve of hub 28 is preferably selected to match the curve of an access opening in the bone to help anchor the device within the bone. Either one or both hub configurations may be used in different types of fractures.

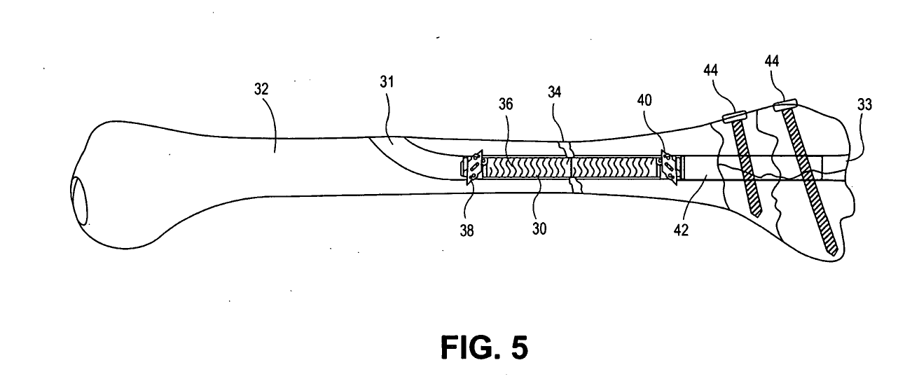

FIG. 5 shows another embodiment of a fracture fixation device 30 inserted into an intramedullary space through an access opening 31 or 33 formed in a fractured bone 32 to span a fracture site 34. Like the device shown in FIG. 3, device 30 has a flexible component 36 formed as a wavy spiral that may be actuated by the compressive forces of an actuator (such as actuator 62 shown in FIG. 6) to become substantially rigid. Device 30 also has one or more pairs of actuatable grippers 38 and 40 and a hub 42. Bone screws 44 have been inserted through bone 32 and hub 42 to stabilize the device within the bone and to fix any bone fragments to the device and to the rest of the bone.

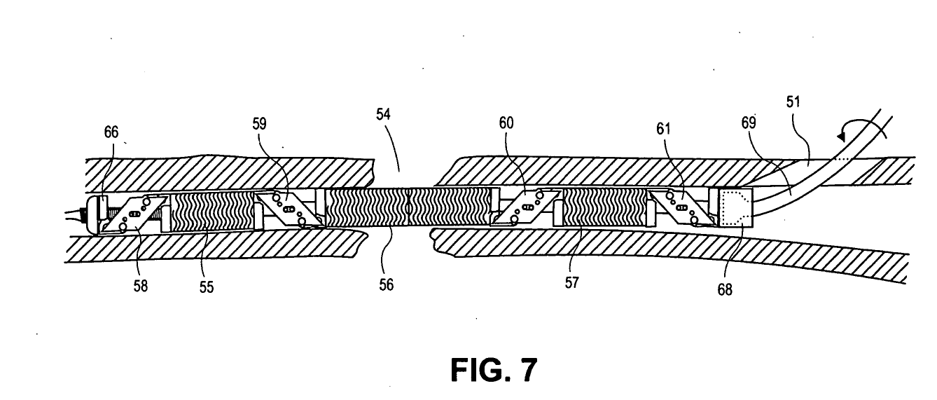

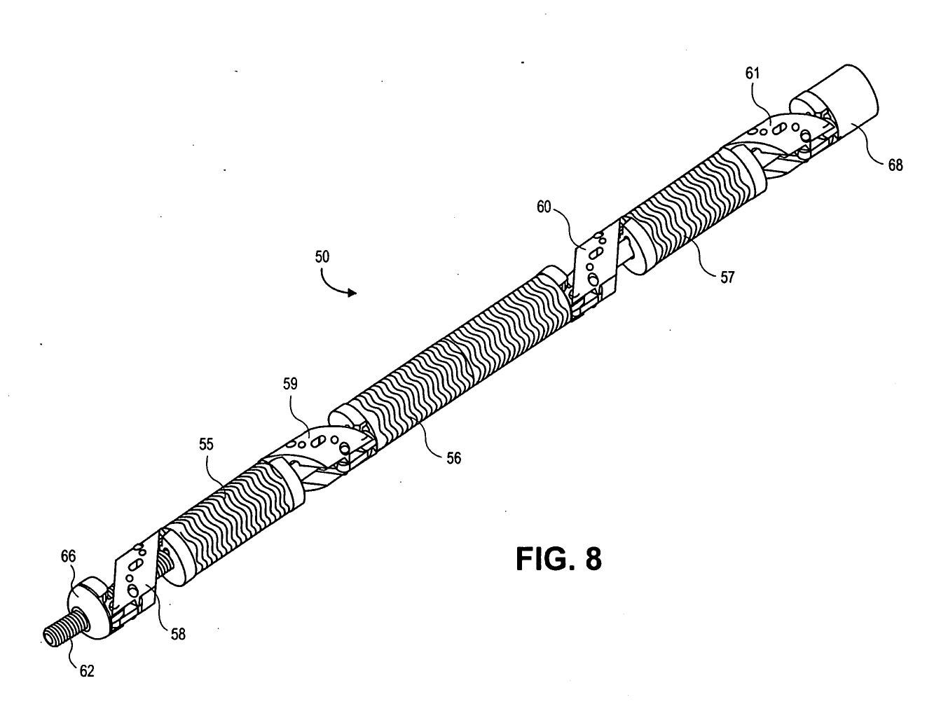

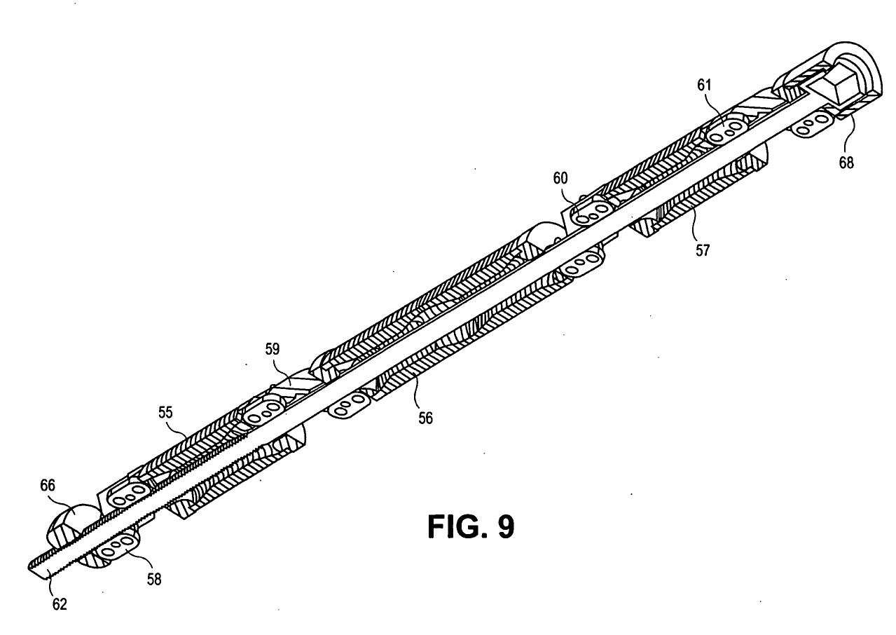

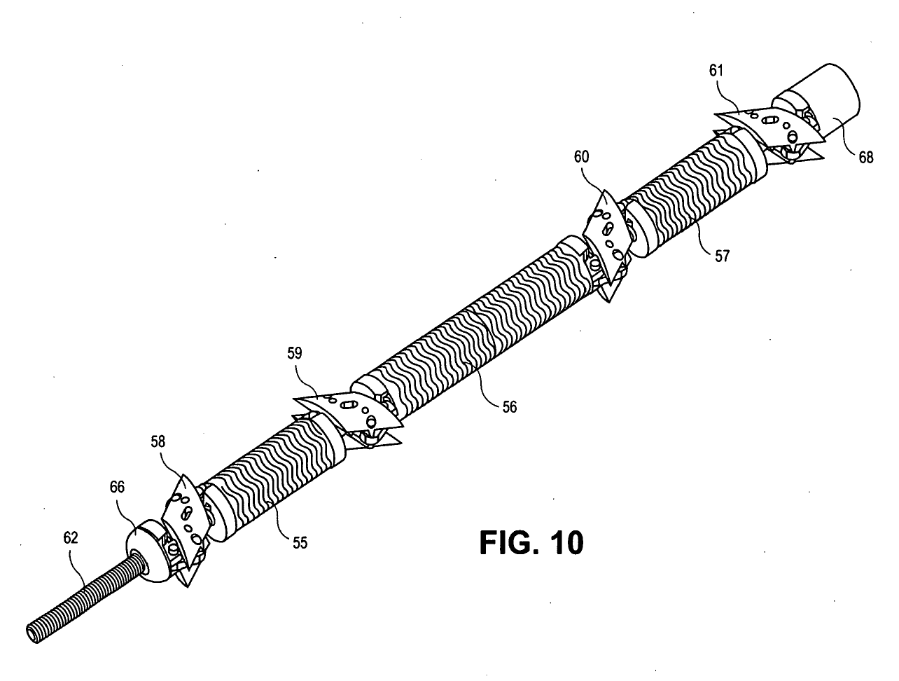

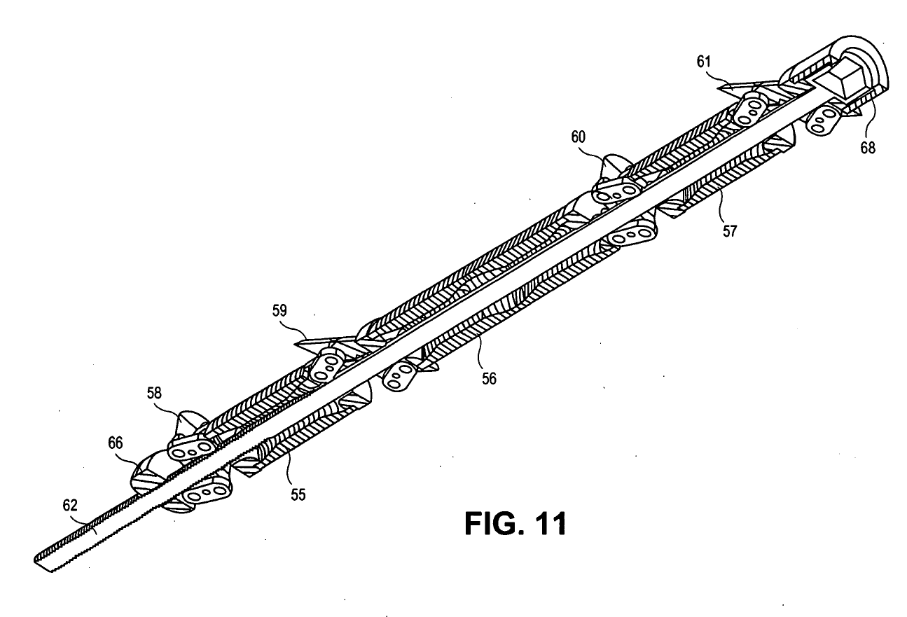

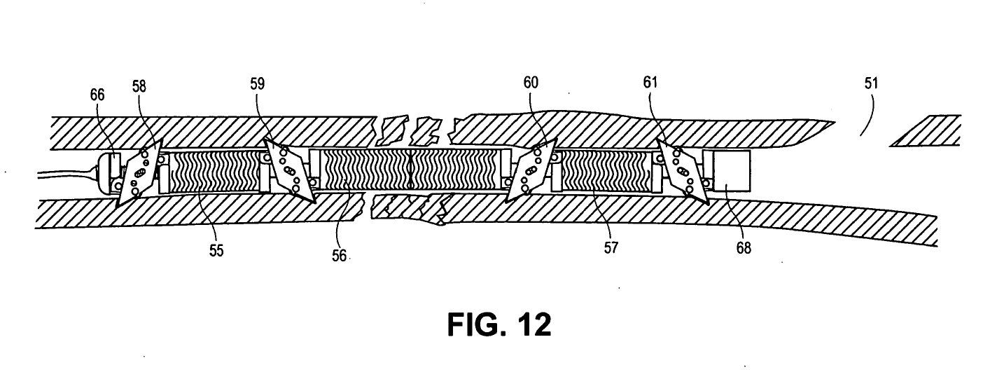

FIG. 6 shows yet another embodiment of a fixation device 50 of the invention in the process of being inserted through an opening 51 into a fractured bone 52 to span a fracture site 54. As shown in additional detail in FIGS. 7-12, device 50 has three actuatable flexible-to-rigid components 55, 56 and 57 and four actuatable grippers 58-61. At one end, device 50 has an actuator 62 with a blunt end 64 to help guide the device within the bone and to push aside any soft bone material within the bone 52. Actuator 62 is threaded and passes through an internally threaded head 66 disposed proximal to the grippers and flexible components through the interior of device 50 to a distal screw head 68. By rotating screw head 68 and actuator 62 (by using, e.g., a flexible screw drive 69, as shown in FIG. 7), threaded head 66 travels distally with respect to the grippers and the flexible-to-rigid components while hub 68 remains stationary. This action foreshortens device 50 to deploy grippers 58-61 and to rigidize components 55-57. When deployed to the configuration shown in FIGS. 10-12, grippers 58-61 tilt outward to dig their tips into the interior surface of the bone, as shown in FIG. 12. To reposition or remove device 50 from bone 52, the actuator may be rotated in the other direction to release the grippers and to permit the flexible-to-rigid components to become flexible again.

In FIGS. 3-12, the flexible-to-rigid members such as 16, 30, 56 and the rigid members such as hub 42 that traverse the fracture are designed with substantially larger external diameters than the rest of the assembly. The larger diameter will limit the amount of bone in-growth that the fracture may experience and thus leave a passage large enough for the ease removal of the rest of the components such as the grippers 60 and 58.

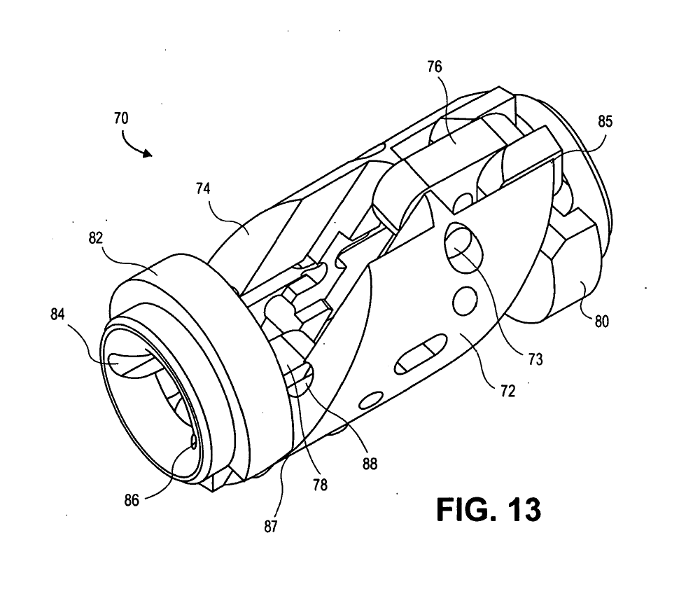

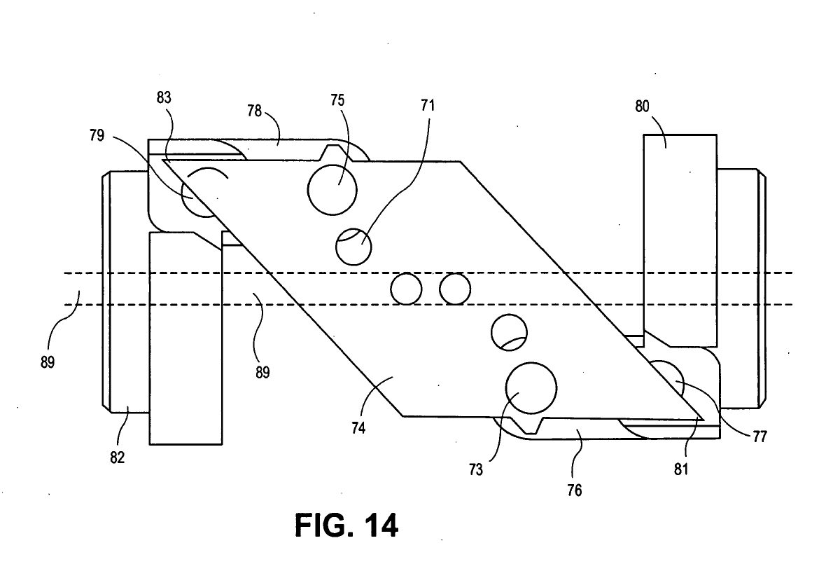

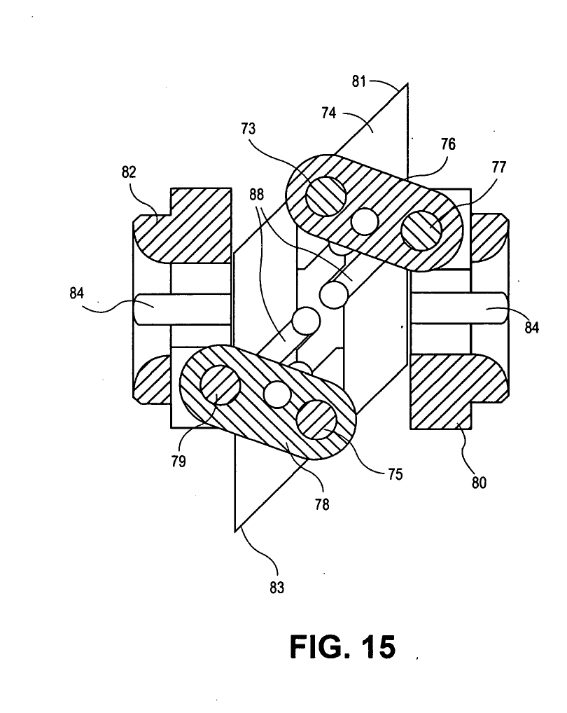

FIGS. 13-15 show details of an actuatable gripper 70 for use with, e.g., the fracture fixation device embodiments described above. In this embodiment, gripper 70 has two rotatable cams 72 and 74. Cams 72 and 74 are attached by pins 73 and 75 to cam arms 76 and 78, respectively. Cam arms 76 and 78 attach by pins 77 and 79 to flanges 80 and 82, respectively. Flanges 80 and 82 connect with the components on either end of the device. In the undeployed configuration shown in FIGS. 13 and 14, cams 72 and 74 are oriented such that the sharp tips 85 and 87 of cam 72 and the sharp tips 81 and 83 of cam 74 do not extend from the cylinder of the gripper. When foreshortened during deployment, however, movement of flanges 80 and 82 toward each other causes cam arms 76 and 78 to rotate about pins 77 and 79 with respect to flanges 80 and 82 and causes cams 72 and 74 to rotate about pins 73 and 75 with respect to cam arms 76 and 78 so that the sharp tips swing out from the cylinder of the gripper, as shown in FIG. 15. Thus, when part of a fracture fixation device that has been inserted into a bone, deployment of the gripper 70 causes the sharp tips of the cams to dig into the bone to anchor the device. An alternative design combines cams 72 and 74 into one integral component.

In order to prevent inadvertent deployment of the gripper, one or more optional lock wires may be inserted into the gripper. As shown in FIG. 13, lock wire channels 84 and 86 may be formed in flanges 80 and 82, and corresponding channels may be formed in flange 80. Likewise, lock wire channels may be formed in the cams, such as channel 88 formed in cam 74, to line up with the lock wire channels formed in the flanges when the gripper is in its undeployed configuration, thereby permitting a lock wire 89 to be inserted through the gripper, as shown in FIG. 14. Lock wire 89 must be removed before the gripper can be rotated to its foreshortened deployed configuration, as shown in FIG. 15. A lock wire may also be inserted across the gripper through holes 71.

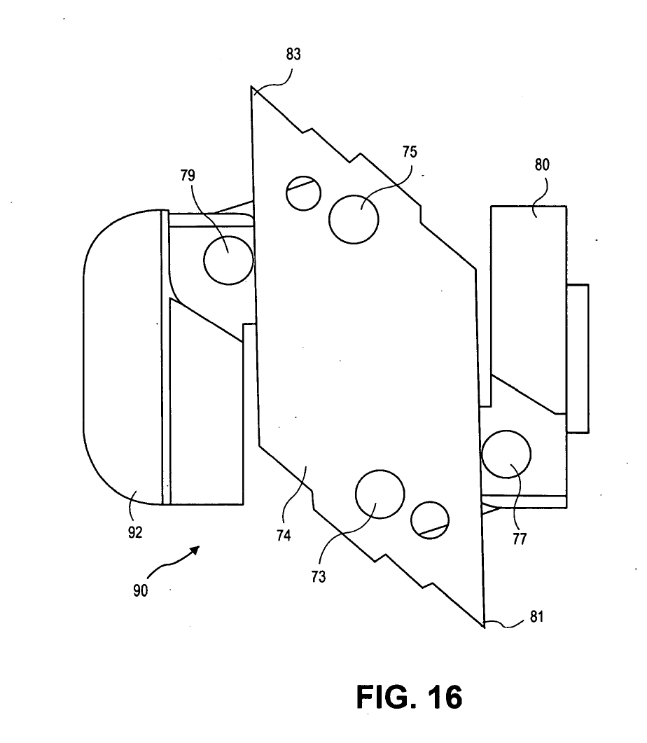

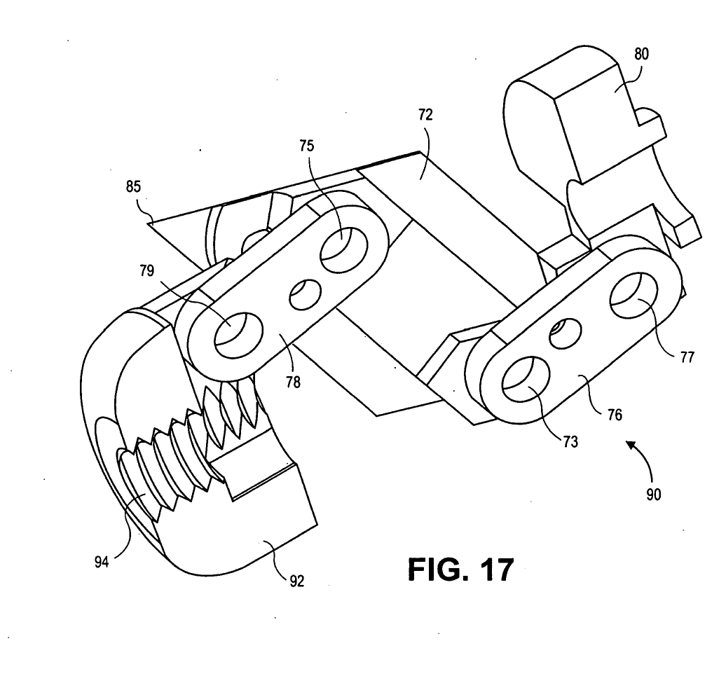

FIGS. 16-18 show a gripper 90 for use on one end of an actuatable fracture fixation device according to one embodiment of the invention. In this embodiment, a threaded flange 92 replaces flange 82 of the earlier gripper embodiment. Internal threads 94 in flange 92 interact with a threaded actuator, such as actuator 62 shown in FIGS. 6-12, for use in foreshortening during deployment.

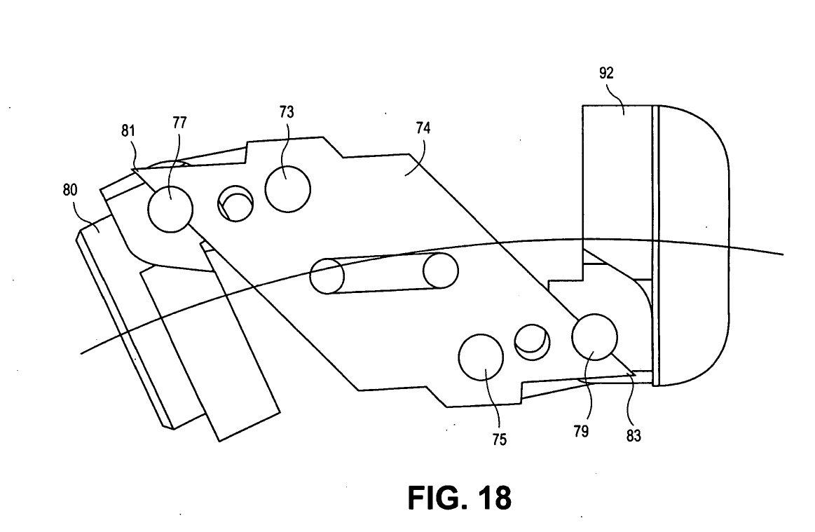

FIG. 18 also demonstrates an advantage of the grippers 70 and 90 shown in FIGS. 13-18. During insertion into the interior of a bone along a curved insertion path, grippers 70 and 90 can adapt to the curve of the insertion path, as shown by the curved line in FIG. 18.

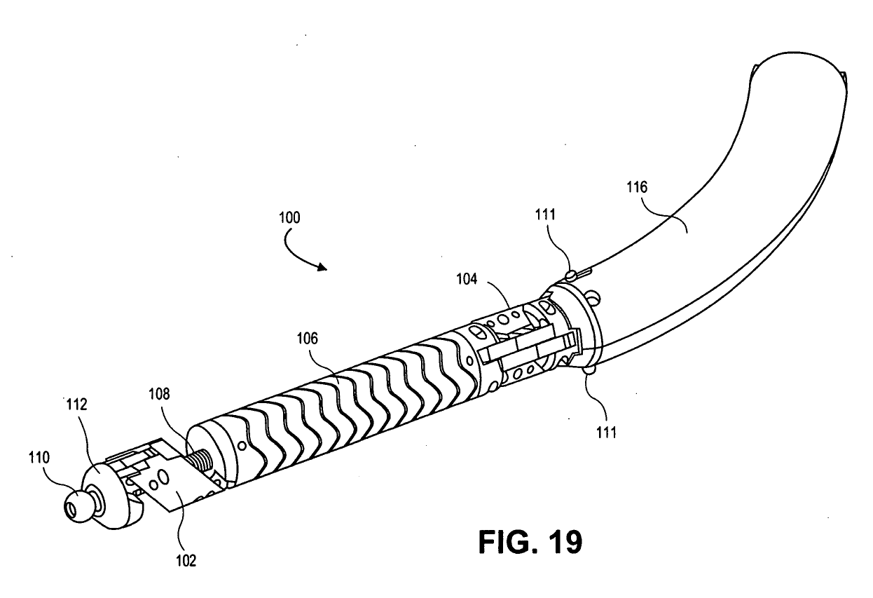

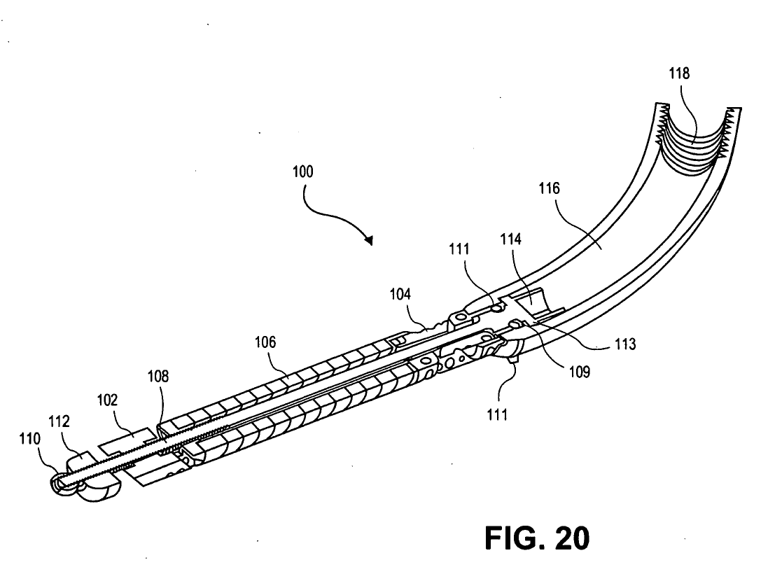

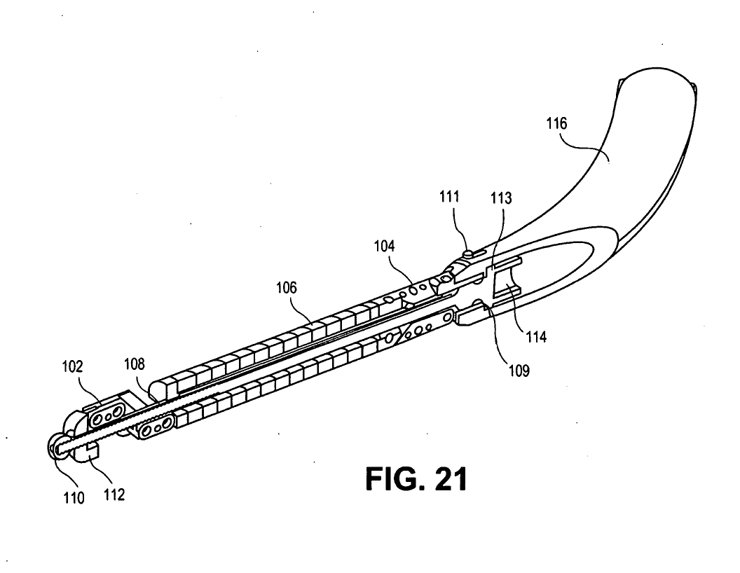

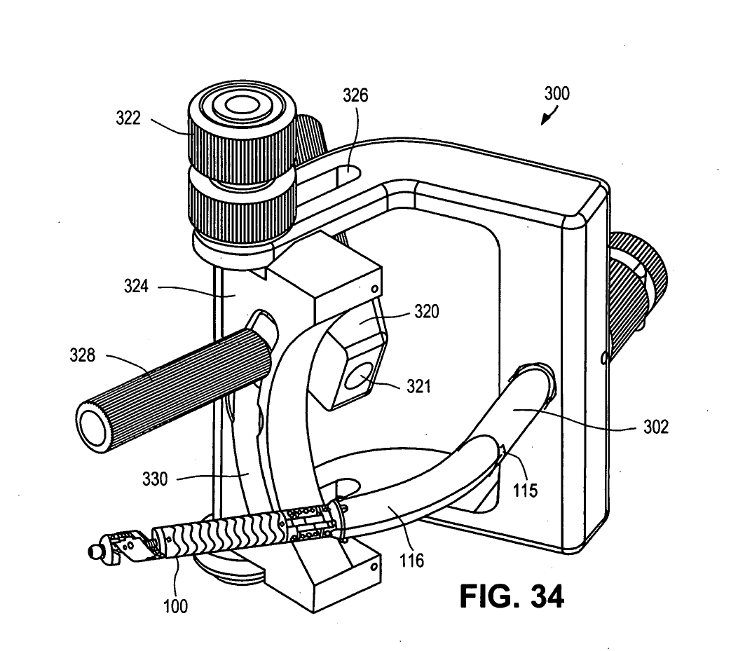

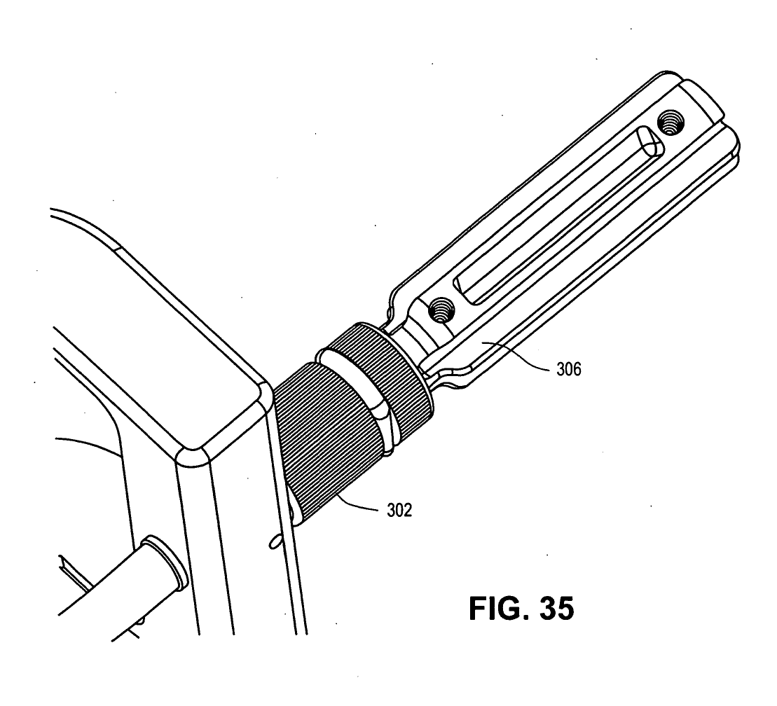

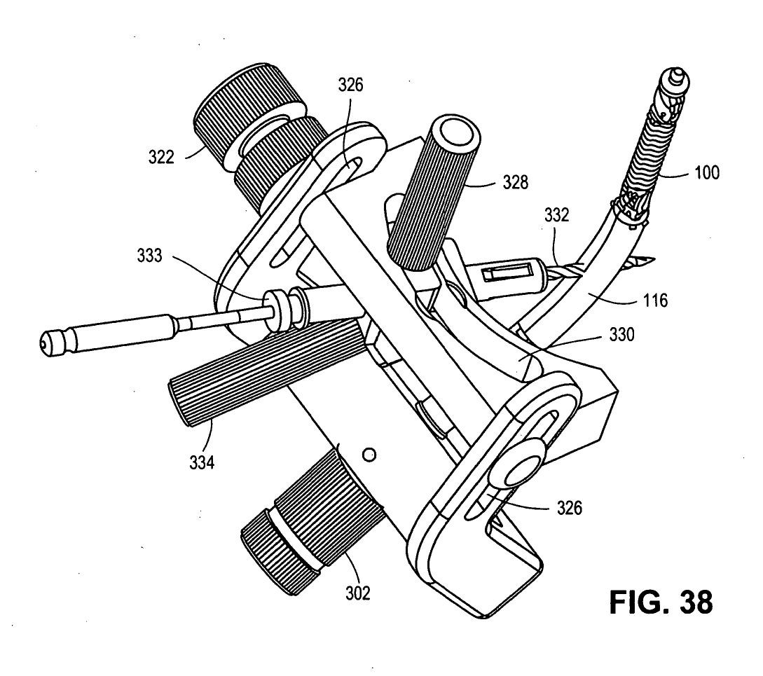

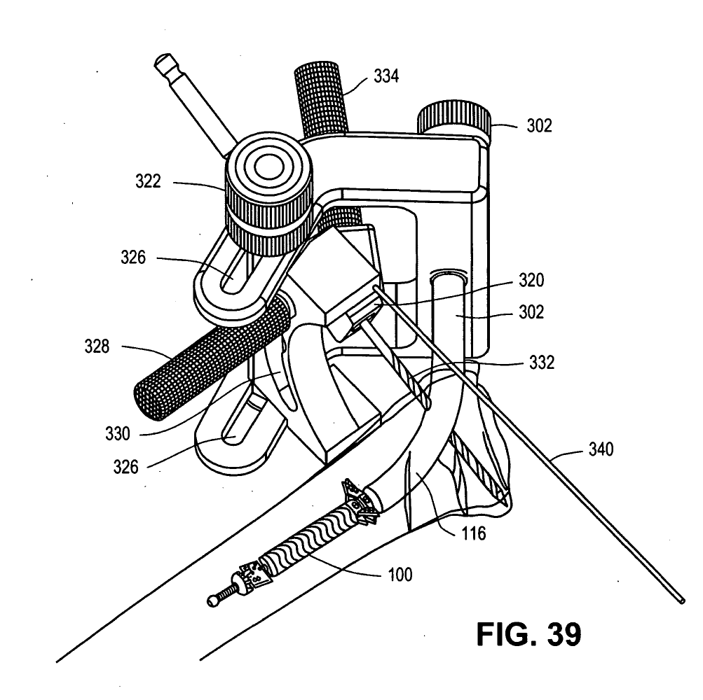

FIGS. 19-21 show yet another embodiment of a fracture fixation device 100 according to the invention. In this embodiment, device 100 has a first gripper 102 constructed, e.g., like the grippers described above with respect to FIGS. 13-18, and a second gripper 104. Extending between grippers 102 and 104 is a flexible-to-rigid body 106. A threaded actuator 108 with a blunt end 110 extends through grippers 102 and 104, body 106 and an internally threaded head 112. A tool engagement feature 114 extends from one end of actuator 108 to enable a screw driver or other tool to rotate actuator to actuate, foreshorten and rigidize fixation device 100. A curved hub 116 is attached to the device distal to gripper 104. Pins 111 secure hub 116 axially to the actuator and device while still permitting the actuator and/or device to rotate with respect to the hub. A flange 113 formed in tool engagement feature 114 engages a lip 109 formed on the inside of hub 116 to transfer any loads from the actuator directly to the hub without overloading pins 111. Internal threads 118 in hub 116 provide for attachment to a deployment tool (such as, e.g., tool 300 shown in FIGS. 34-41) or for the insertion of a plug (not shown) after deployment of fracture fixation device 100 within a fractured bone. Hub 116 that transverses the fracture is designed with a larger external diameter than the rest of the device. This features in the hub 116 limits, during the healing process of the fracture, the amount of bone in-growth and calluses that would otherwise prevent the removal of the device. The external diameter of the hub 116 is preferably also tapered to facilitate the release and removal of the device. The larger diameter of the hub 116 during withdrawal leaves behind an opening larger than the rest of the device such as the grippers 104, 108 and flex-to-rigid body 106 and thus facilitates the removal of the device.

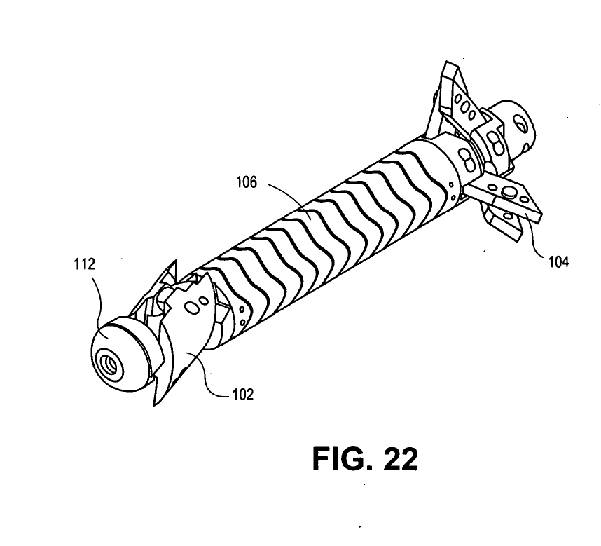

FIG. 22 shows device 100 of FIGS. 19-21 without hub 116, actuator 108 and blunt end 110. As shown, grippers 102 and 104 have been actuated to a deployed configuration.

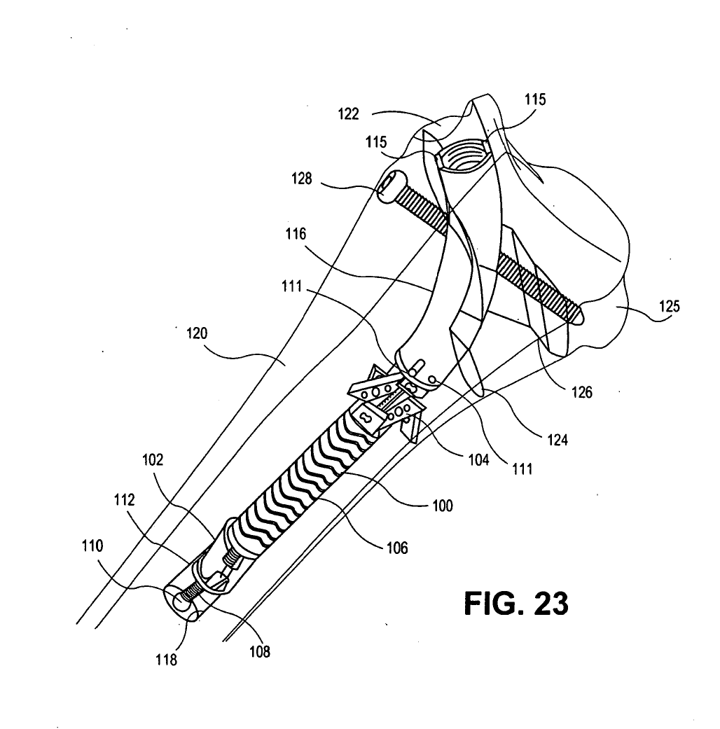

FIGS. 23-25 show fracture fixation device 100 of FIGS. 19-22 deployed within a space 118 formed in a bone 120. Device 100 has been inserted through an opening 122 formed in a bony protuberance of bone 120, and the grippers 102 and 104 have been actuated to grip the interior of the bone. Appropriate tools (such as those discussed below) have been used to form space 118 with a curved distal portion extending proximally from opening 122 to a substantially straight proximal portion through one or more fracture areas, such as fracture lines 124 and 126. As shown, hub 116 is disposed within the curved portion of space 118 while flexible-to-rigid body 106 and the grippers 102 and 104 are disposed in the substantially straight portion of space 118. During delivery to space 118 in the device's undeployed configuration, grippers 102 and 104 and body 106 are substantially flexible so as to accommodate the curve of the distal portion of the opening. After actuation, however, the device body 106 becomes rigid through the compression and interaction of its segments during foreshortening and deployment of grippers 102 and 104.

In this embodiment, hub 116 is substantially rigid and has a curve approximating that of the curved portion of opening 118. In some embodiments of the method of this invention, some or all of hub 116 is placed on one side of a bone fracture while the remainder of the fracture fixation device is placed on the other side of the fracture.

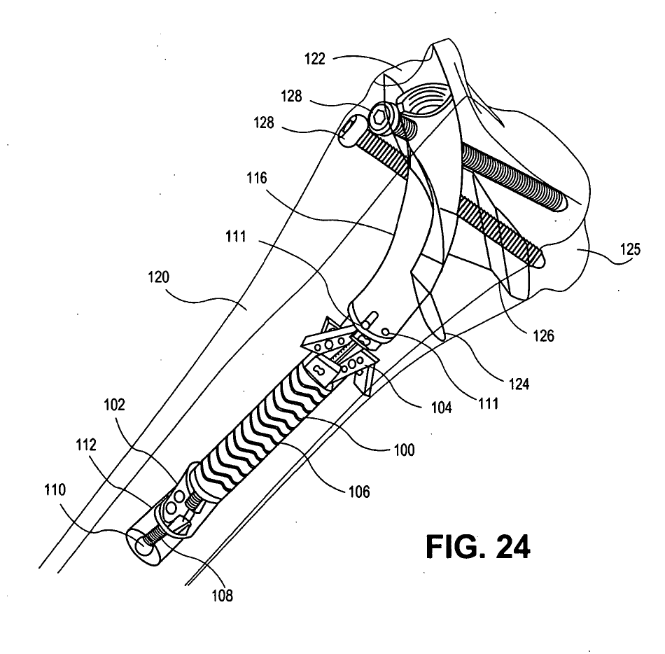

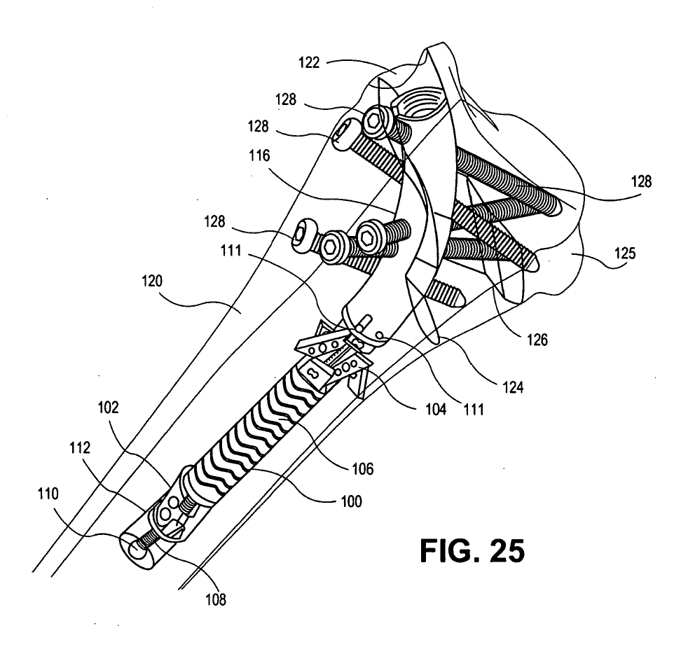

In some embodiments, hub 116 is made of PEKK or PEEK implantable grade material and may be injection molded. Using the tools of this invention, a hole through bone 120 may be drilled at any angle and through any portion of hub to permit a screw to be inserted through the bone and fixation device. In FIG. 23, one screw 128 has been inserted through hub 126 to help anchor device 100 within the bone and to hold bone fragment 125 to the main portion of the bone. In FIGS. 24 and 25, multiple screws 128 have been inserted in various positions and orientations. These figures illustrate the ability to place screws wherever needed and at whatever orientation required.

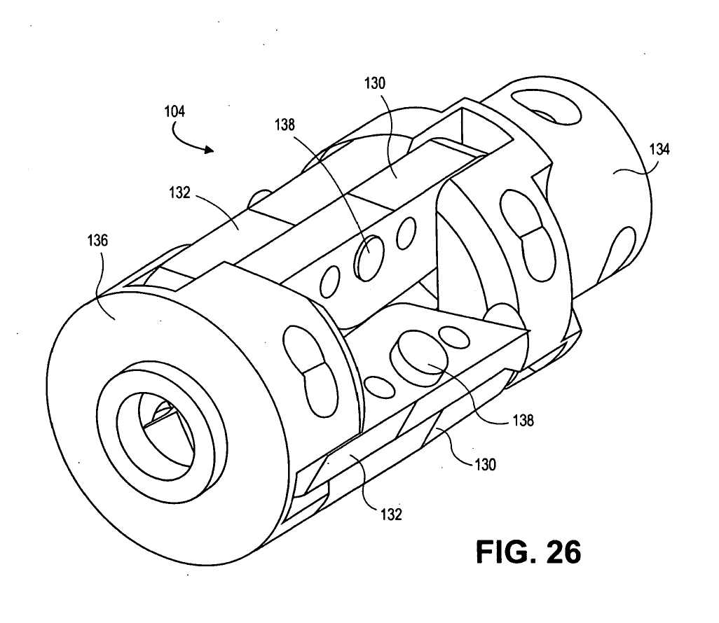

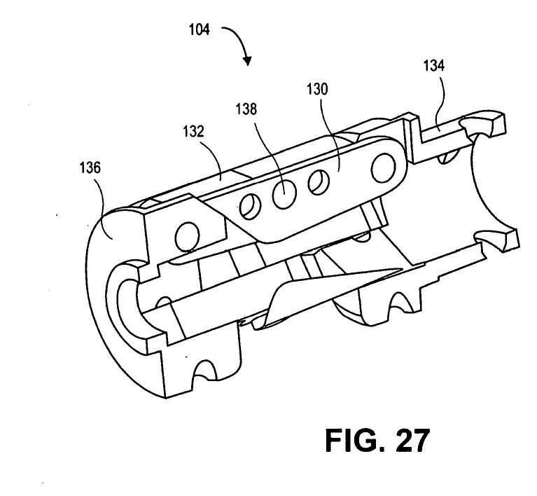

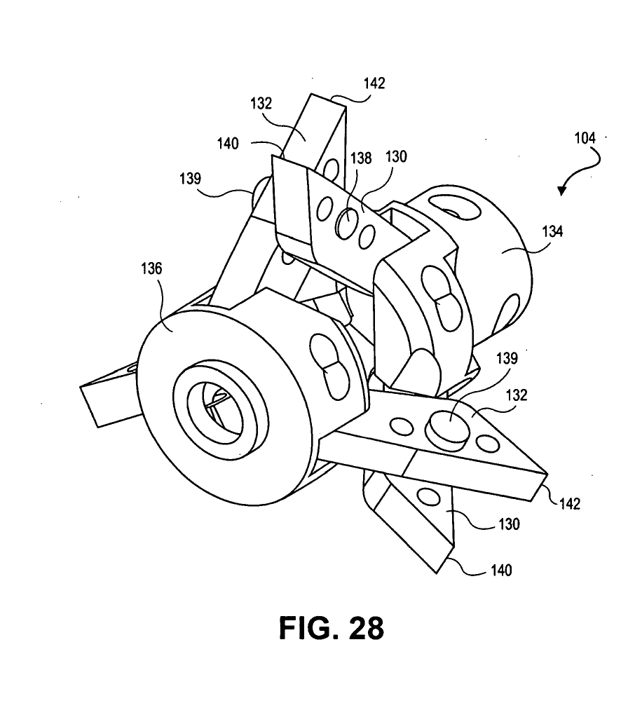

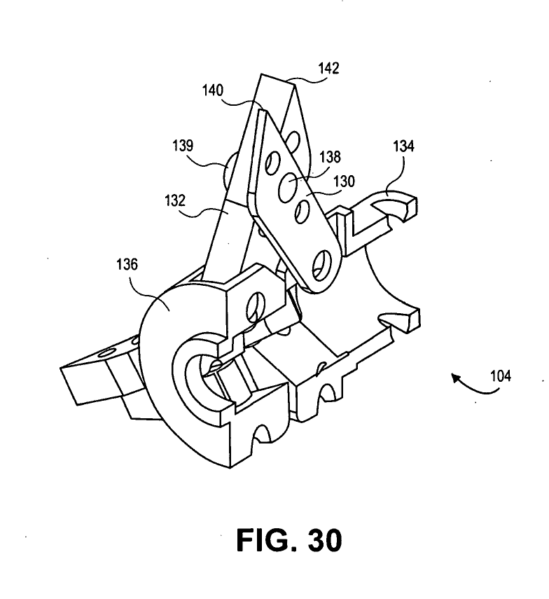

FIGS. 26-31 show details of a gripper 104 for use with a fracture fixation device, such as the devices of FIGS. 1-5, 6-12 or 19-25. FIGS. 26 and 27 show gripper 104 in an undeployed configuration. FIGS. 28-31 show gripper 104 in deployed configurations. Gripper 104 has three sets of anchor elements, with each set including a first anchor leg 130 and a second anchor leg 132. Anchor leg 130 is connected to flange 134 and extends toward flange 136, and anchor leg 132 is connected to flange 136 and extends toward flange 134. Legs 130 and 132 are rotatably connected by a pin 138 that is welded to leg 130. Leg 132 rotates freely about pin 138. A larger head portion 139 on pin 138 keeps leg 132 rotatably mounted on pin 138. Alternatively, a washer may be added to pin 138 at the end opposite of the head 139. The pin 138 may then be welded to the washer instead of leg 130. In this arrangement the washer and head 139 retains pin 138 within the holes in leg 132 and leg 130 such that the pin and washer may rotate freely within both legs 130 and 132. This washer arrangement provides a lower stress concentration on the weld, which can result in a more reliable connection in some embodiments.

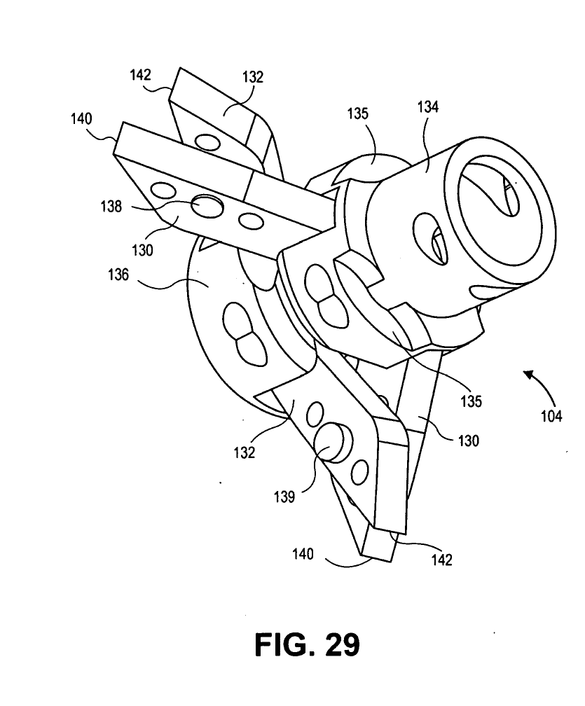

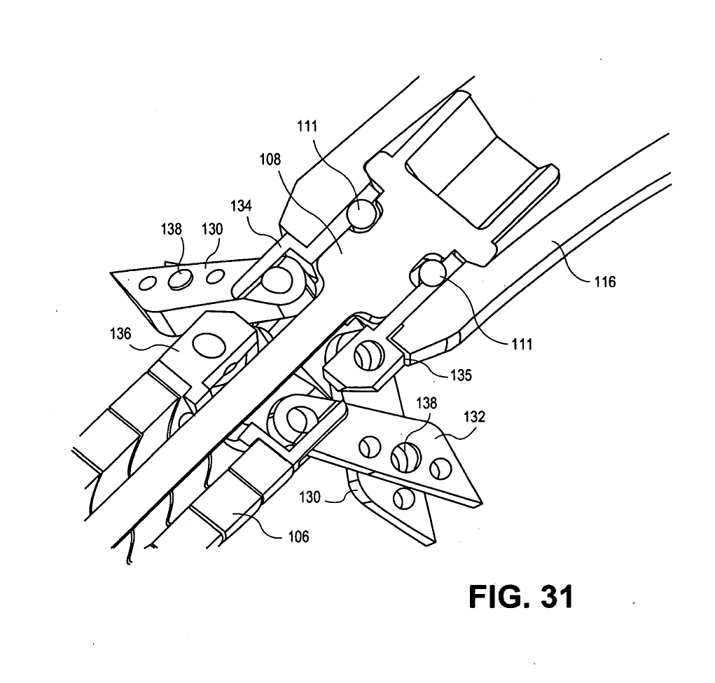

In the undeployed configuration of FIGS. 26 and 27, legs 130 and 132 lie substantially parallel to each other within the cylinder of flanges 134 and 136. When the fracture fixation device is actuated by, e.g., turning an actuator 108 to foreshorten the device (as shown in FIG. 31), flanges 134 and 136 are moved closed together. This movement causes the outer edges 140 and 142 of legs 130 and 132, respectively, to rotate outward to grip the inside surface of the bone in a deployed configuration, as shown, e.g., in FIGS. 23-25. Movement of flanges 134 and 136 away from each other retracts legs 130 and 132 toward and into their undeployed configuration for repositioning and/or removal of the device from the bone. Cut-outs 135 formed in gripper 104 mate with corresponding shapes in the hub to provide a rotational keying feature enabling the transmission of torque from the hub to the rest of the device without overloading the pins connecting the hub to gripper 104, as shown in FIGS. 29 and 31.

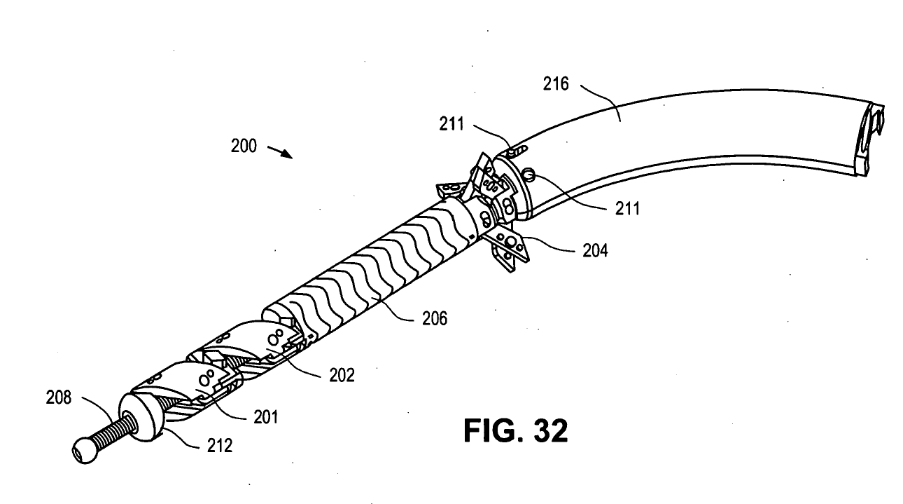

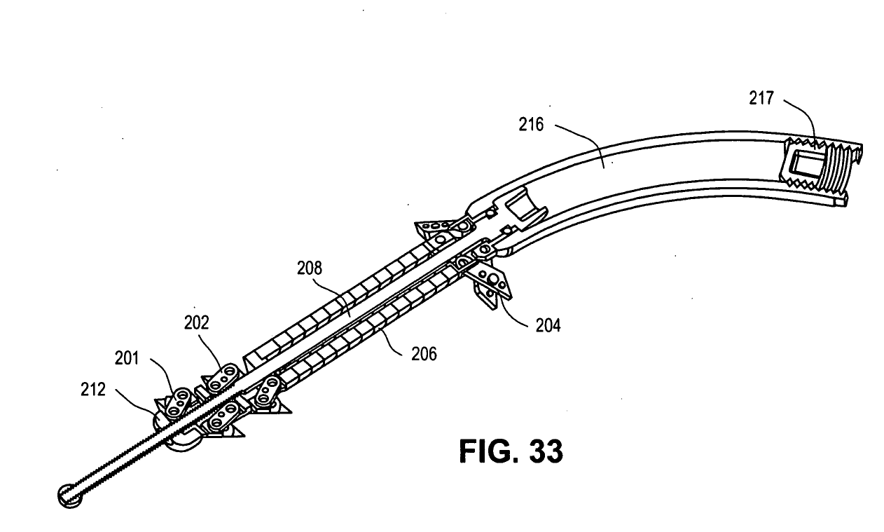

FIGS. 32 and 33 show yet another embodiment of a fracture fixation device according to the invention. Like the embodiment shown in FIGS. 19-31, device 200 has a curved hub 216, a distal gripper 204 (formed, e.g., like gripper 104 of the prior embodiment), a flexible body 206 and two proximal grippers 201 and 202 (each formed, e.g., like gripper 102 of the prior embodiment). Pins 211 attach hub 216 to gripper 204. As in the embodiment of FIGS. 19-31, a threaded actuator 208 cooperates with an internally threaded head 212 to foreshorten and actuate the device. The sectional view of FIG. 33 shows a threaded plug 217 that has been inserted into the distal opening of hub 216 to seal the device after actuation.

As seen from the discussion above, the devices of this invention can be easily modified by adding grippers or by placing grippers in different positions on the device to address fractures where more gripping forces are needed.

FIGS. 34-39 show a deployment tool 300 for use with a fracture fixation device 100 of this invention. As shown in FIG. 34, the hub 116 of fixation device 100 connects to a stem 302 of tool 300. Hub 116 may be provided with, e.g., connection features 115 for this purpose, as shown, e.g., in FIG. 23. This connection orients fixation device 100 with tool 300. In particular, tool 300 aids in the use of a fixation device actuation tool and alignment of a drill with the fixation device's hub after the device has been deployed within a fractured bone and in the insertion of screws into the hub and bone.

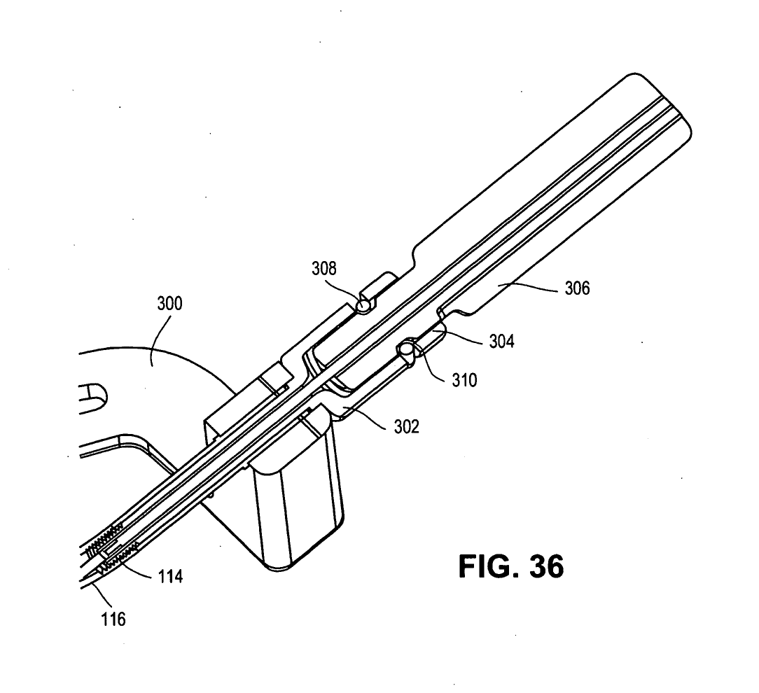

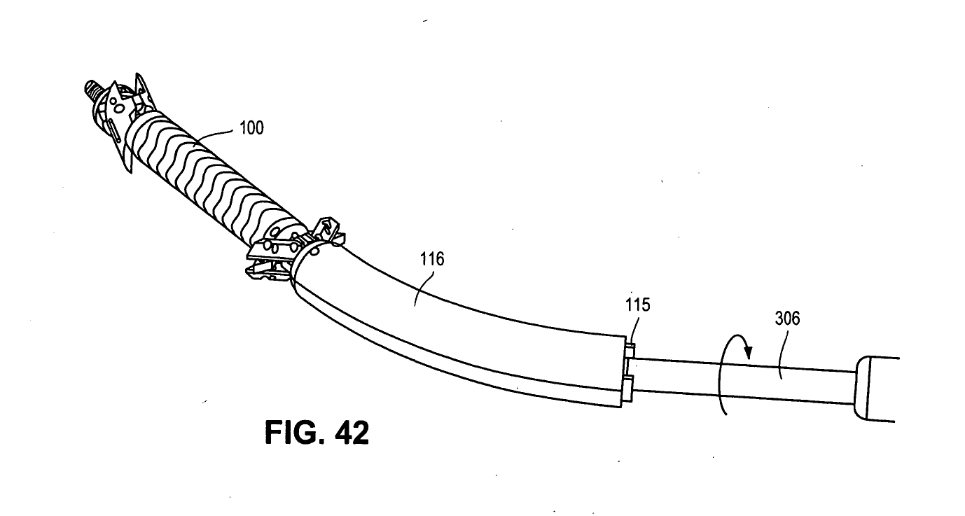

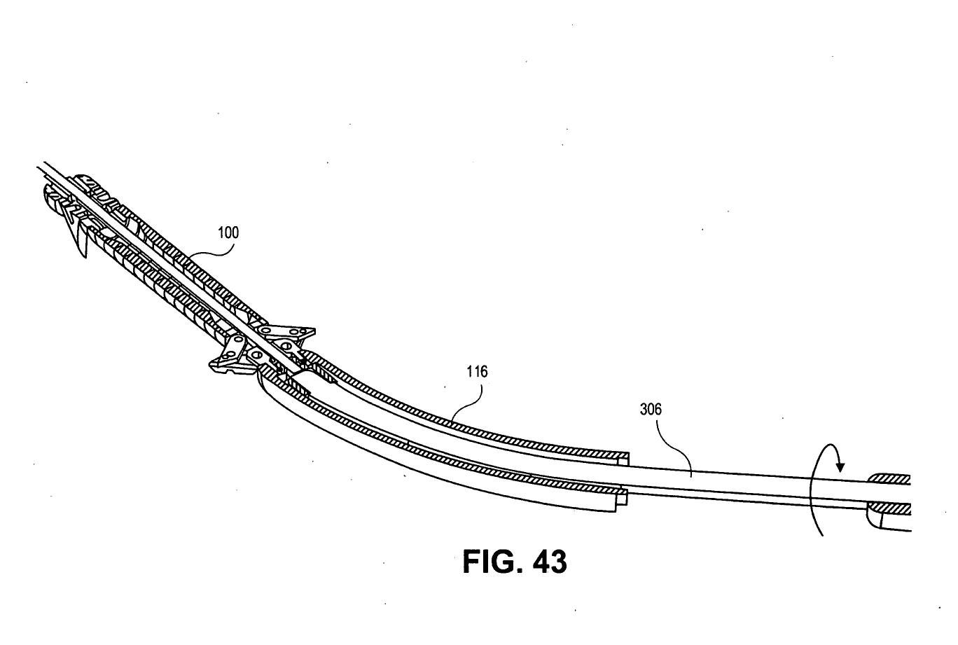

Access to the interior of fixation device 100 is provided by a port 304 through stem 302 so that, e.g., a flexible screw driver 306 may be inserted through hub 116 to device actuator 108, as shown in FIG. 36. Rotation of flexible screw driver 306 and actuator 108 moves device 100 from an undeployed configuration to a deployed configuration, as shown in FIGS. 42 and 43 (which show the interaction of flexible screw driver 306 and fixation device 100 outside of tool 300). A flexible ring 308 may be provided to interact with a groove 310 formed in flexible screw driver 306 to provide proper axial positioning between the screw driver and the tool engagement feature 114 of actuator 108 while still permitting the flexible screw driver to rotate.

Tool 300 also helps orient a drill and enables it to find the hub of the fixation device even when the fixation device is inside the bone and cannot be seen by a user. When fixation device 100 is properly attached to stem 302, the bore 321 of drill guide 320 points toward the device's hub 116 even when the drill guide is rotated along curved guide 300 or translated along grooves 326. In order to provide the user with flexibility in drill placement (e.g., in order to place one or more screws through hub 116 as shown in FIGS. 23-25), tool 300 permits drill guide 320 to be moved with respect to stem 302 and attached hub 116. Drill guide 320 may be translated proximally and distally with respect to hub 116 by loosening knob 322 and moving support 324 along grooves 326. In addition, drill guide 320 may be rotated about hub 116 by loosening knob 328 and moving drill guide 320 along curved groove 330.

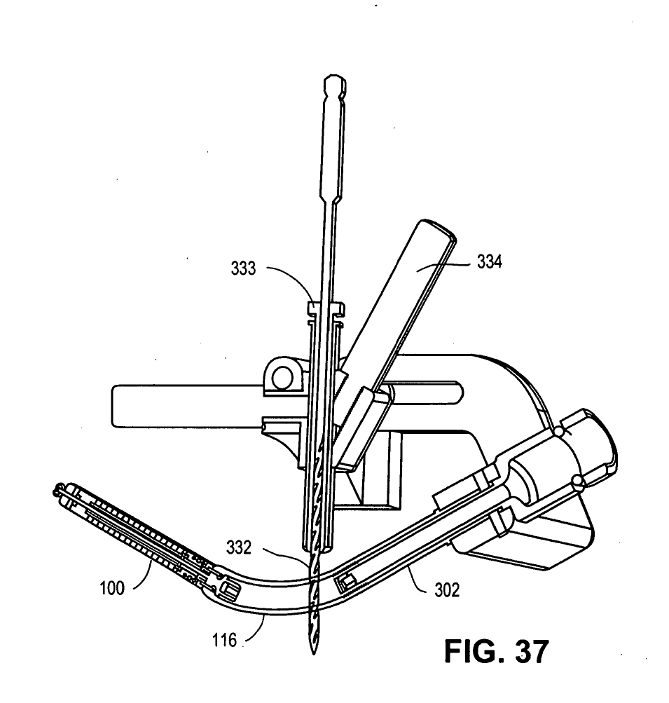

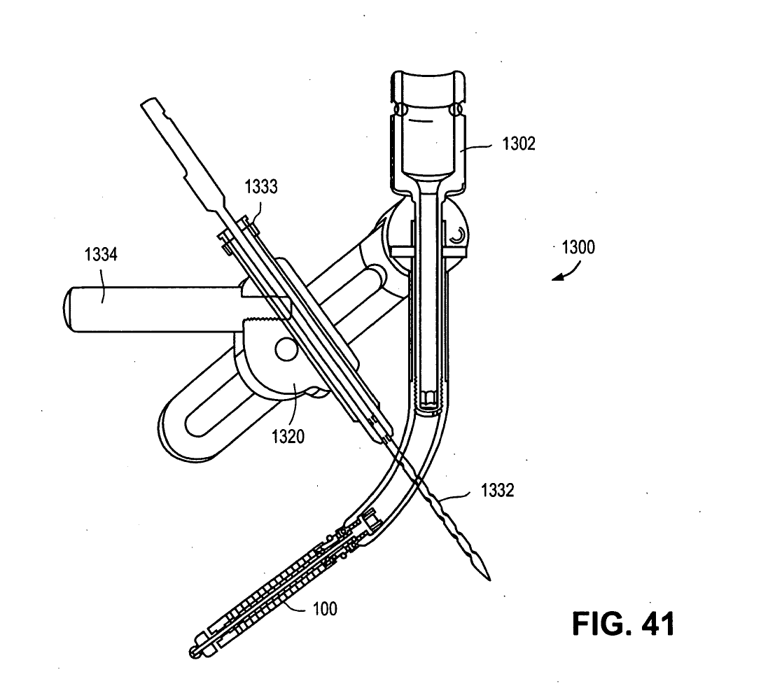

FIGS. 37-41 show tool 300 being used to guide a drill 332 toward and through hub 116 (and through the bone, as shown in FIG. 41). The drill sleeve 333 surrounding drill bit 332 is held in place within bore 321 by a set screw 334. An external x-ray visible aim 340 may extend from drill guide 320 to show on x-rays the orientation of the drill bit 332 within the patient's bone, as shown in FIG. 41. The drill bit may be provided with a scale to show depth of the drilled hole and, therefore, the length of the screw needed. In some embodiments, the drill 332 may have a sharp tip to reduce skittering of the drill against the device hub and/or bone during drilling. The tip included angle may be less than 100° and preferably between 25° and 35° to ensure penetration of the hub.

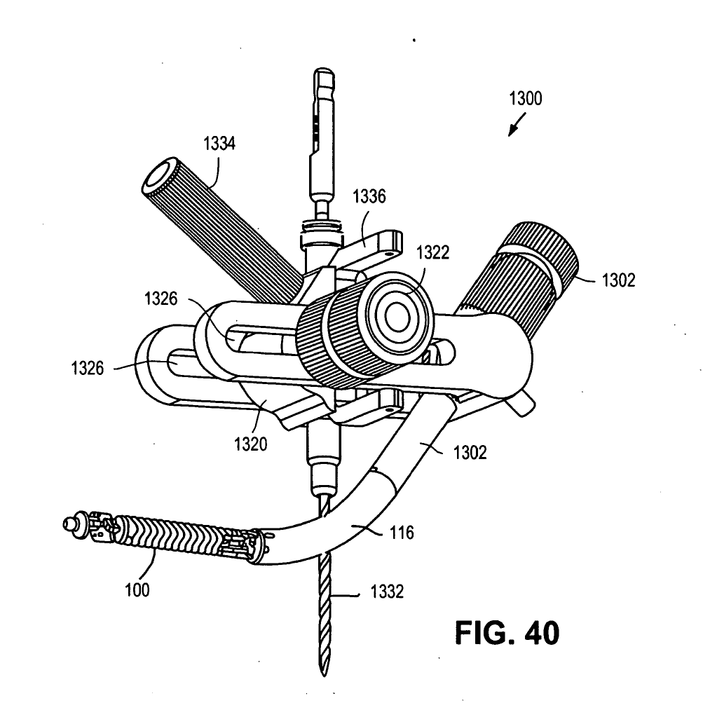

FIGS. 40-41 show an alternative planar tool 1300 being used to guide a drill 1332 toward and through the hub 116 of a fracture fixation device. As with the earlier embodiment, access to the interior of fixation device 100 is provided by a port 1304 through a stem 1302 so that, e.g., a flexible screw driver may be inserted through hub 116 to device actuator 108. A flexible ring 1308 may be provided to interact with a groove formed in the flexible screw driver to provide proper axial positioning between the screw driver and the tool engagement feature 114 of actuator 108 while still permitting the flexible screw driver to rotate.

Like the deployment tool described above, tool 1300 also helps orient a drill and enables it to find the hub of the fixation device even when the fixation device is inside the bone and cannot be seen by a user. When fixation device 100 is properly attached to stem 1302, the bore of drill guide 1320 points toward the device's hub 116 even when the drill guide is translated along grooves 1326 or is rotated above the axis of knob 1322. In order to provide the user with flexibility in drill placement (e.g., in order to place one or more screws through hub 116 as shown in FIGS. 23-25), tool 1300 permits drill guide 1320 to be moved with respect to stem 1302 and attached hub 116. Drill guide 1320 may be translated proximally and distally with respect to hub 116 by loosening knob 1322 and moving support drill guide 1320 along grooves 1326. In addition, drill guide 1320 may be rotated about stem 1302 and hub 116.

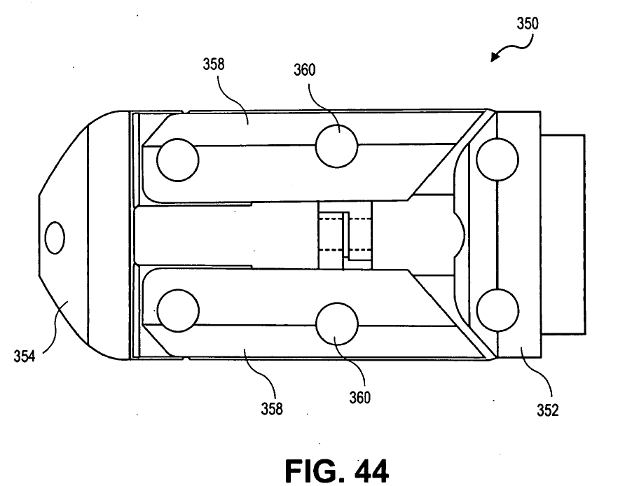

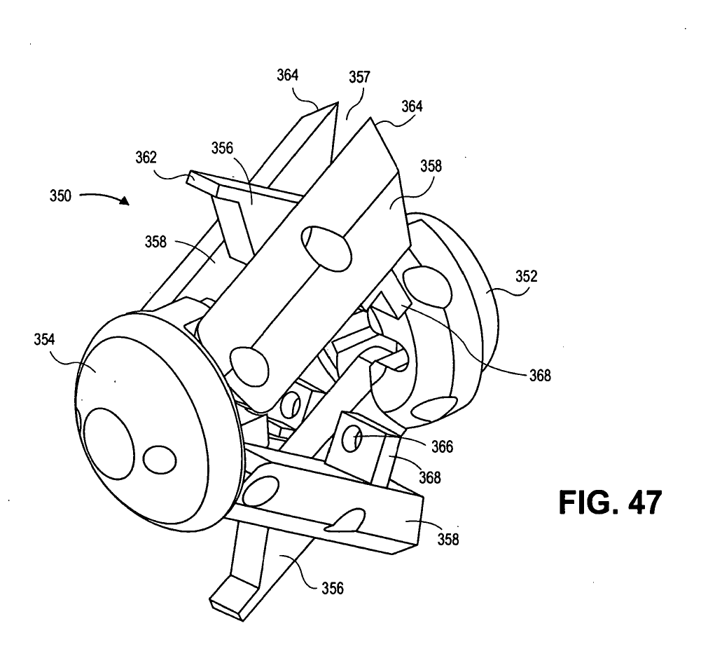

FIGS. 44-48 show another embodiment of a gripper 350 for use with a fracture fixation device. As shown, gripper 350 is designed to be used on the leading end of the fixation device. It should be understood that this gripper could be used at other points in the fixation device as well.

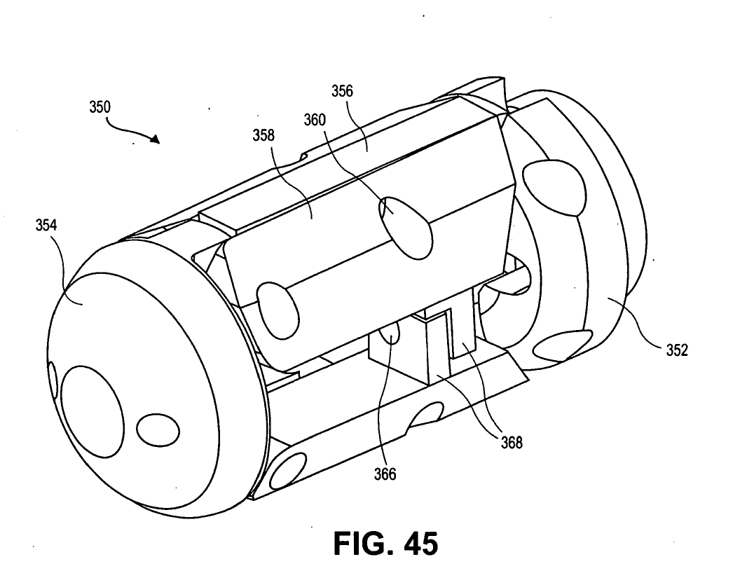

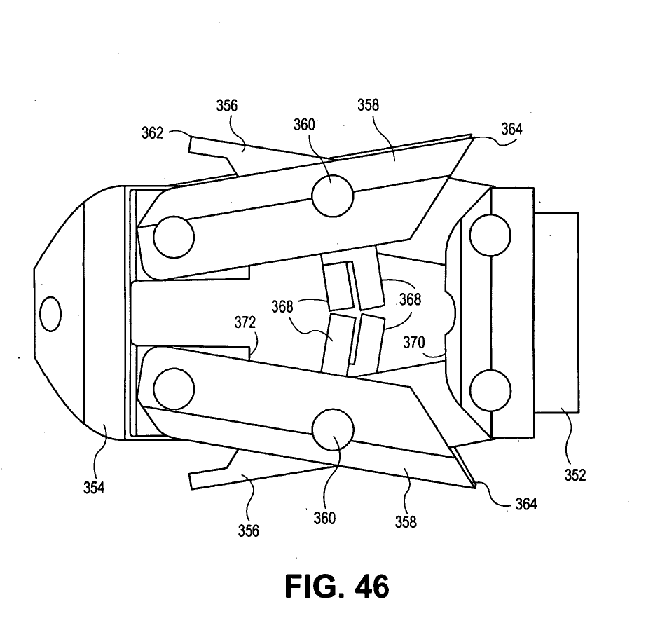

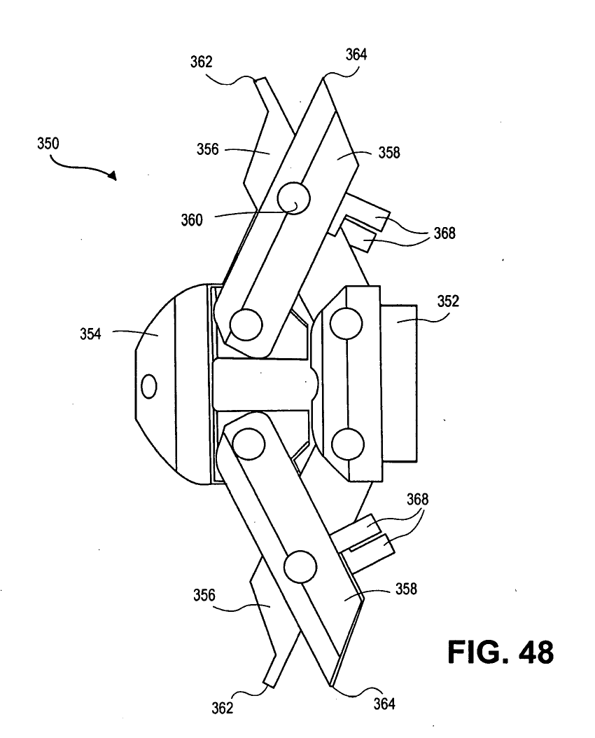

Extending between flange 352 and nose cone flange 354 are two sets of anchor elements. Anchor legs 356 are rotatably attached to flange 352 and extend toward flange 354, and split anchor legs 358 are rotatably attached to nose cone flange 354 and extend toward flange 352. Anchor legs 356 are disposed in the split 357 of anchor legs 358. Legs 356 and 358 are rotatably connected by a pin 360. In the undeployed configuration of FIGS. 44 and 45, legs 356 and 358 lie substantially parallel to each other within the cylinder of flanges 352 and 354. When the fracture fixation device is actuated by, e.g., turning an actuator to foreshorten the device, flanges 352 and 354 are moved closer together. This movement causes the outer edges 362 and 364 of legs 356 and 358, respectively, to rotate outward to grip the inside surface of the bone in a deployed configuration, as shown, e.g., in FIGS. 46-48. Movement of flanges 352 and 354 away from each other retracts legs 356 and 358 toward and into their undeployed configuration for repositioning and/or removal of the device from the bone. A lock wire (shown in phantom in FIG. 44) disposed in channels 366 formed in projections 368 extending between the two sides of the proximal anchor legs 358 prevents inadvertent actuation of the anchors. Stop surfaces 370 and 372 on flanges 352 and 354, respectively, meet to provide a limit to extension of gripper 350, as shown in FIG. 48.

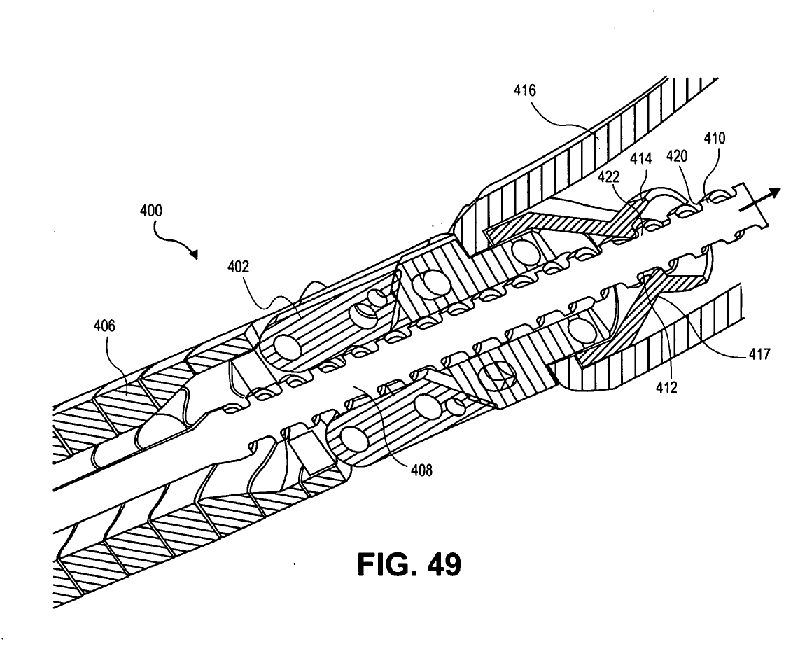

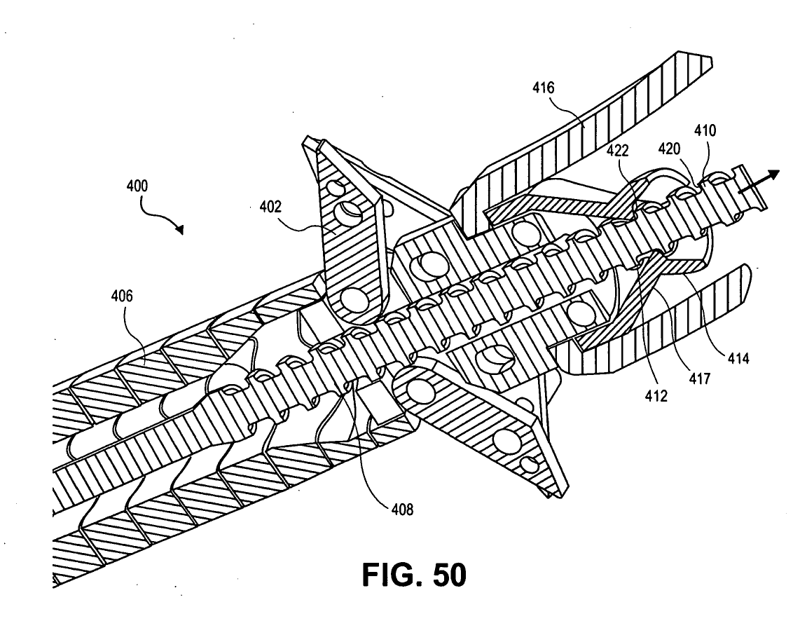

FIGS. 49-50 show another embodiment of a fracture fixation device 400 similar to device 100 shown in FIGS. 19-25 using a distal gripper 402 similar to that shown in FIGS. 44-48. Instead of a threaded rotating actuator, however, device 400 uses a ratcheting actuator 408. Actuator passes through device hub 416, gripper 402 and flexible body 406 to a flange (not shown) at the other end of the fixation device. To foreshorten and actuate the fixation device (thereby extending the grippers and rigidizing the flexible body), actuator 408 is tensioned by pulling in the direction of the arrow in FIG. 49. As it moves distally, ridges 410 formed in actuator push against a cam surface 412 formed in crown 414 in ratchet 417, expanding the crown enough to permit the ridges to pass through. After passing through the crown, surface 420 of ridge 410 meets a stop surface 422 of ratchet crown 414, thereby preventing proximal movement of actuator 408 after it has been tensioned. After deployment of the fracture fixation device within a fractured bone, the portion of actuator 408 extending from the end of the device after suitable tensioning may be cut and removed. A tool (not shown) may be used to release the ratchet in the event fixation device 400 must be repositioned or removed.

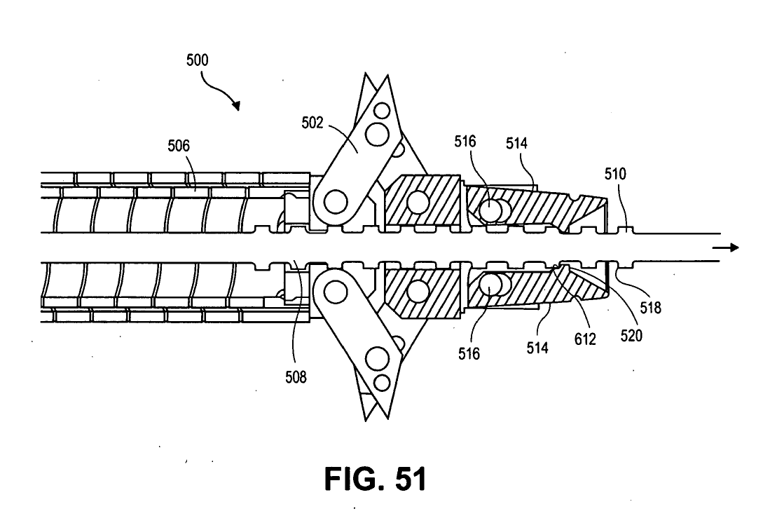

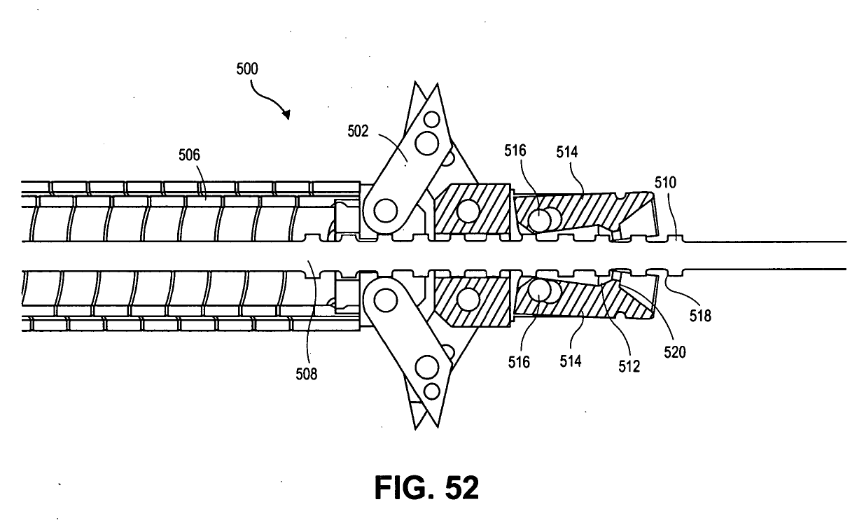

FIGS. 51-52 show yet another embodiment of a fracture fixation device 500 similar to that of FIGS. 49-50. Flexible body has two concentric tubular members 506A and 506B with opposing clockwise/counterclockwise helical cuts. In order to rigidize flexible body 506 and deploy gripper 502, actuator 508 is tensioned in the direction of the arrow in FIG. 51. As it moves in that direction, ridges 510 of actuator 508 move against cam surfaces 512 of ratchet members 514, which rotate outwardly around pins 516. The interaction of face 518 of ridge 510 with face 520 of ratchet member 514 prevents actuator 508 from moving back the direction it came. The actuator 508 may be released from the ratchet by using a tool (not shown) to move the ratchet members 514 to the position shown in FIG. 52.

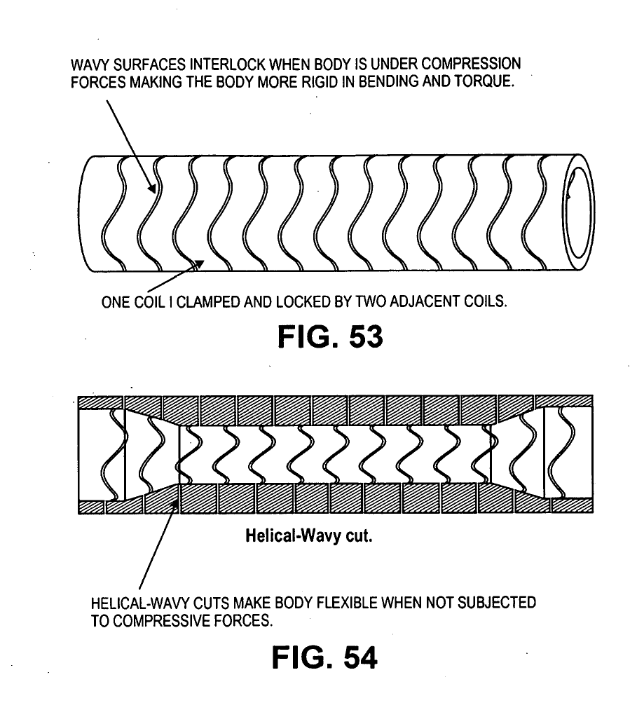

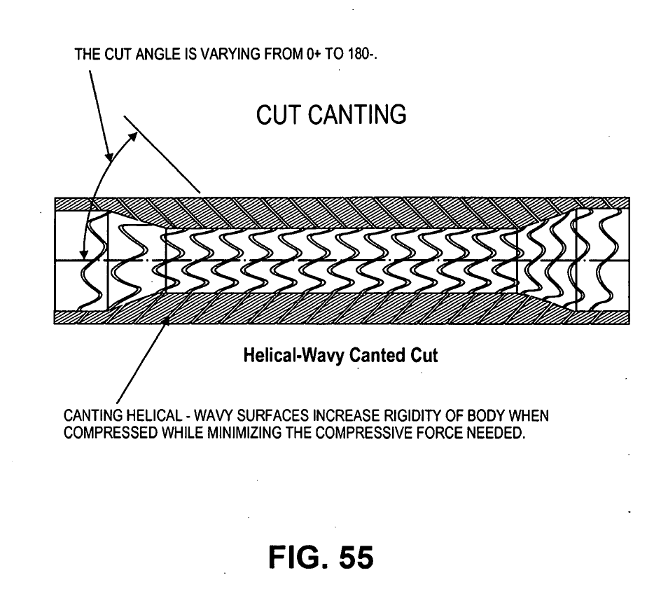

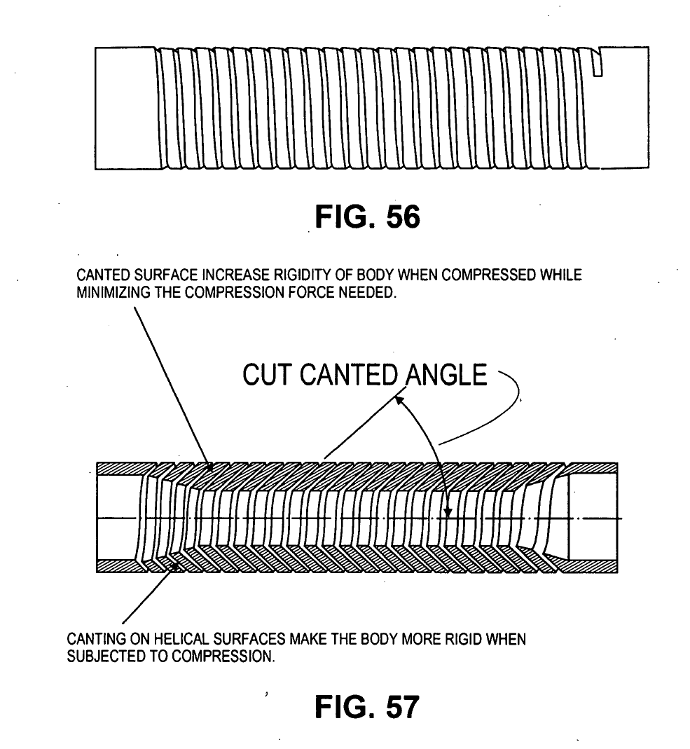

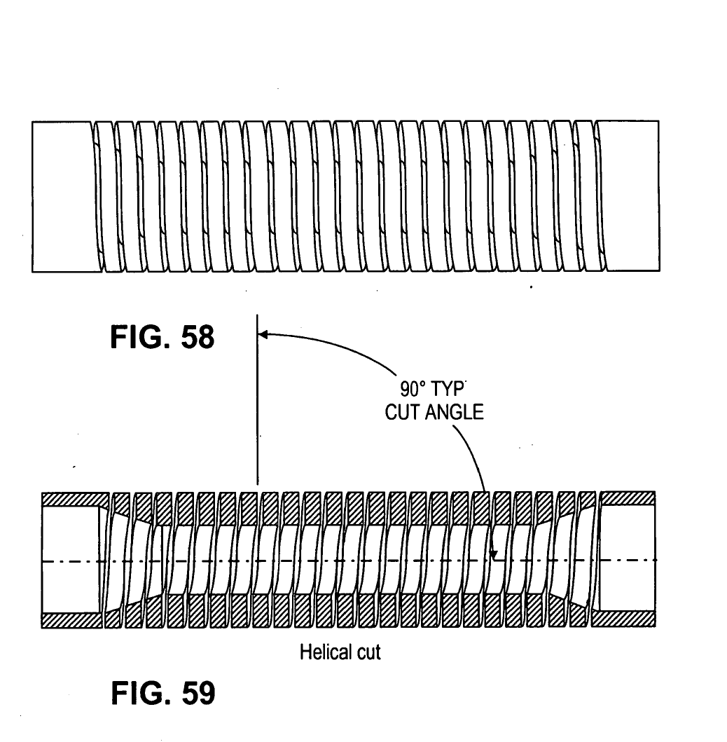

FIGS. 53-59 show alternative designs for the flexible body of a fracture fixation device according to this invention. FIGS. 53 and 54 show helical wavy cuts formed in the flexible-to-rigid body so that it is flexible when not compressed and rigid when foreshortened and compressed. FIG. 55 shows a canted helical wavy cut formed in the flexible-to-rigid body. FIGS. 56 and 57 show canted angles formed in the helical cuts of the flexible-to-rigid body. FIGS. 58 and 59 show a helical cut to form the flexible-to-rigid body. As in the earlier embodiments, the shape of the helical turns enables the transmission of bending and torque along the flexible-to-rigid body, in addition to the rigidizing function the body performs.

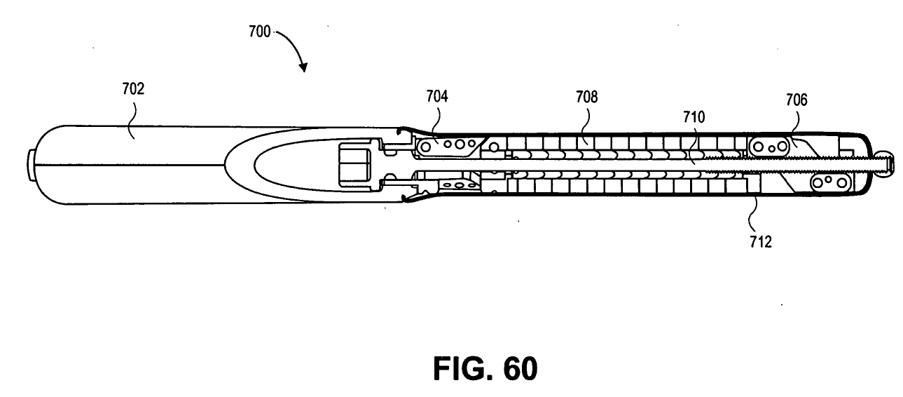

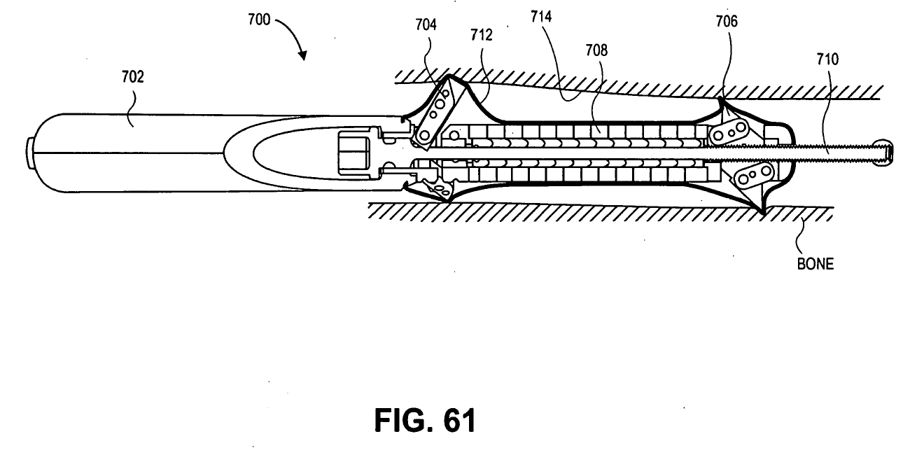

FIGS. 60 and 61 show an embodiment of a fracture fixation device having a flexible membrane cover. Like some earlier embodiments, fixation device 700 has a hub 702, expandable and releasable grippers 704 and 706 and a flexible-to-rigid body 708. A threaded actuator 710 extends through the device. A tubular flexible membrane 712 covers the grippers and flexible-to-rigid body and conforms to the device's outer shape. As shown in FIG. 61, when the fixation device is actuated to extend grippers 704 into the interior space 714 formed in a bone and to rigidize body 708, the flexible membrane 712 stretches and forms a tent-like envelope that keeps debris and bone ingrowth away from the device, thereby facilitating subsequent removal of the device from the bone. The flexible membrane can be used with any of the previously described embodiments of the fracture fixation device, or with similar implanted devices as well, with or without grippers.

The membrane may serve one or more other purposes instead of or in addition to facilitating removal of a device that has been implanted in the body for a period of time. For example, the membrane may provide corrosion resistance to the implanted device, and/or may reduce inflammation caused by the implanted device. The membrane may deliver therapeutic agents inside the body over a predetermined period of time, such as for the treatment of the central nervous system. The membrane may also comprise, contain or otherwise deliver biologically active material or proteins that provide some benefit to the body, anatomy, immunological, or biochemical response by the patient. For example, a bioactive agent may be used for the stimulation of bone growth or the prevention of infection. Immunological agents may be used for the treatment of immuno-deficiencies. In some embodiments, the membrane may comprise matter that prevents or inhibits bone ingrowth. The membrane may also contain radio-opaque material, such as barium sulfates or other metals, to allow the membrane to be seen on x-rays or with other imaging. This can be useful for checking the integrity of the membrane after implanting and/or to determine if it has migrated.

The flexible membrane may be made from a thermoset or thermoplastic material. Exemplary materials that may be used include, thermoplastic elastomer TPE, silicon rubber, Teflon®, PTE or flexible PTFE. Inert polyethylene, polypropylene or other long term biocompatible materials such as PEEK or PEKK may be used for the membrane. Additional materials suitable in some embodiments, and that may be used alone or in combination with other materials include polymeric materials such as Dacron (the general category polyester), polyolefin (the general category of straight chain carbon polymers such as polypropylene, polyethylene), polysilanes (the general category of silicon backbone polymers), polymers of aromatic hydrocarbon backbone (polycarbonates, polysulfones, polymers that contain a benzene ring), epoxide type polymers that are thermoset or thermoplastic (the general class of polymers that contain the strained carbon-oxygen-carbon bridge), hydrogels, ionomers, and metalocenes. Inorganic materials that include calcium salts, calcium containing ceramics, calcium phosphates, and hydroxyapitite may also be used. Inorganic materials that include heavy metals such as palladium, cobalt 60, iridium, platinum, and strontium for nuclear medical treatments may be used. Therapeutic agents loaded into any of the above are contemplated, including Warfarin, opiates for pain, pharmaceuticals for red cell, white cell, and platelet production, T-cell enhancement pharmaceuticals, and bone morphogenic proteins.

Depending on the application, the materials described above may form the membrane substrate itself, may be co-extruded or co-molded with the membrane material(s), and/or may be coated on the inner or outer surface of the membrane. The membrane can be formed from a dipping process. In the dipping bath the above constituents may be present. In some embodiments, the material may be a liquid, gel, powder or other form of material contained within the membrane. The membrane can be configured to be porous to allow any material contained therein to pass through the membrane at a predetermined rate, or the membrane can be configured to be generally impervious. Pore sizes may range from 0.1 nanometers to 100 microns in some embodiments.

In some embodiments, the thickness of the membrane is between about 0.010 inches to about 0.030 inches, before deployment of any grippers, depending on the size and nature of the fracture fixation device it covers. The membrane may stretch and become thinner in some regions when grippers are deployed. In other embodiments, the membrane thickness may be between about 0.01 microns and about 5 milimeters. In some of these embodiments, the membrane is not thick enough to be self-supporting, but rather is a coating over the implantable device. The membrane thickness need not be uniform, but may be made thicker is some regions. For instance, the regions around the grippers may be made thicker to prevent or inhibit the grippers from puncturing the membrane when expanded against the bone. A radius or chamfer or a blunt tip may be provided at the gripping points of the gripper's arm to also prevent or inhibit puncturing the membrane. Depending on the load required by the bone fixation, in some embodiments, it may be desirable to allow the grippers to penetrate the membrane in order to obtain a better hold between the grippers and the bone.

The membrane may be formed by injection molding, liquid injection molding, transfer molding, extrusion, or other suitable manufacturing method such as overmolding on the fixation device and grippers. The membrane may be closed or semi-closed at one or both ends, such as the semi-closed distal end shown in FIGS. 60 and 61. In other words, the distal end of actuator 710 may be covered by membrane 712, or may protrude through membrane 712 as shown. One or both ends may be open, such as the open proximal end shown in FIGS. 60 and 61. The membrane may be configured to cover the entire device, or just one or more portions of the device.

Various methods may be employed to secure membrane 712 to fixation device 700. In some embodiments, membrane 712 may be secured by being stretched over a portion of device 700, and/or held in place by an adhesive. In some embodiments a tie, strap, snap ring, clamp, sleeve, or similar element may surround one or more portions of membrane 712 and device 700. A groove may also be provided in device 700, as shown in FIGS. 60 and 61, to receive a portion of membrane 712 and/or a securing element. A snap ring or similar retaining element may be molded into the membrane during fabrication to aid in assembly. An otherwise open end of a membrane may extend beyond an end of device 700 and tied or otherwise secured to itself. An end of a membrane may wrap around an end of the device and be secured within and interior cavity of the device, such as with a press-fit plug.

In some embodiments, multiple membranes may be provided, one over the other(s), for redundant layers in case one or more membranes are ruptured. Extra layers may be provided over the entire protected area of the device, or in just limited areas such as the grippers. Multiple layers or varying membrane thicknesses may also be useful in controlling diffusion gradients across the membrane. Multiple membrane sleeves may only partially overlap each other, each covering one end or portion of the fixation device. Multiple layers may be stretched over one another, thermally bonded together, and/or secured in place with adhesive. The membrane and device may be configured to provide a hermetic seal around all or a portion of the implanted device to prevent or impede material ingress and egress, or the membrane may merely surround the device to inhibit bone ingrowth.

It is envisioned that the membrane and fixation device may be provided in an operating room as a single, preassembled unit. This unit may be provided in a pre-sterilized condition, or be ready for sterilization just prior to the surgical procedure it is to be used in. Alternatively, the fixation device and membrane may be provided in a surgical kit as separate units in the same or separate packaging. The fixation device and membrane may then be assembled, either before or after sterilization, if not already sterilized before packaging.

While various embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A bone fixation device comprising:
an elongate body;
an actuateable gripper disposed on the elongated body;
an actuator operably connected to the gripper to deploy the gripper from a retracted configuration to an expanded configuration; and
a membrane surrounding at least a portion of the elongate body or the gripper.

2. The bone fixation device of claim 1 wherein the device comprises a first state in which at least a portion of the body is flexible and a second state in which the body portion is generally rigid.

3. The bone fixation device of claim 2 wherein the actuator is operably connected to the elongate body to change the elongate body from its flexible state to its generally rigid state.

4. The bone fixation device of claim 2 wherein the membrane covers the portion of the body that may be changed from flexible to generally rigid.

5. The bone fixation device of claim 1 wherein the membrane covers at least a portion of the elongate body and covers the gripper.

6. The bone fixation device of claim 1 further comprising a second expandable gripper, wherein the membrane covers both of the grippers and a portion of the elongate body located between the grippers.

7. The bone fixation device of claim 1 wherein the membrane comprises a porous material.

8. The bone fixation device of claim 1 wherein the membrane comprises a non-porous, generally impervious material.

9. The bone fixation device of claim 1 wherein the membrane comprises biologically active matter.

10. The bone fixation device of claim 1 wherein the membrane comprises matter which provides a therapeutic effect to a portion of the body.

11. The bone fixation device of claim 1 wherein the membrane has a thickness of between 0.010 and 0.030 inches.

12. The bone fixation device of claim 1 wherein the gripper comprises at least one arm having a distal end that moves outwardly to engage the bone when the gripper is in the expanded configuration.

13. The bone fixation device of claim 12 wherein the distal end comprises a sharp point.

14. The bone fixation device of claim 12 wherein the distal end comprises a blunt point.

15. The bone fixation device of claim 1 wherein the gripper comprises at least two arms that pivot in a scissors fashion relative to each other when the gripper is deployed from the retracted configuration to the expanded configuration.

16. The bone fixation device of claim 1 wherein the membrane has a first portion and a second portion, and wherein the first portion has a thickness that is different than a thickness of the second portion when the gripper is in the retracted configuration.

17. The bone fixation device of claim 1 wherein the membrane is overmolded on at least a portion of the elongate body and the gripper

18. The bone fixation device of claim 1 wherein the membrane comprises a retainer element molded therein.

19. The bone fixation device of claim 1 wherein the membrane is elongate and comprises two ends, and wherein the membrane comprises an opening at each of the ends.

20. A surgical kit comprising:
a bone fixation device having an elongate body, an actuateable gripper disposed on the elongated body, and an actuator operably connected to the gripper to deploy the gripper from a retracted configuration to an expanded configuration; and
a membrane configured to surround at least a portion of the bone fixation device.

21. The surgical kit of claim 20 further comprising one or more packages that enclose the bone fixation device and the membrane and maintain them in a pre-sterilized condition.

22. A method of repairing a fracture of a bone, the method comprising:
covering at least a portion of a bone fixation device with a flexible membrane;
inserting the device and the membrane into an intramedullary space of a bone to place a first portion of the device on one side of a fracture and a second portion of the device on another side of the fracture; and
operating an actuator to deploy at least one gripper of the device to engage an inner surface of the intramedullary space to anchor the fixation device to the bone.

23. The method of claim 22 wherein the membrane covers the at least one gripper.

24. The method of claim 22 further comprising the step of converting at least a portion of an elongate body of the device from a generally flexible state to a generally rigid state after the inserting step.

25. The method of claim 24 wherein the converting step comprises operating the actuator.

26. The method of claim 22 further comprising the step of removing the device and the membrane from the intramedullary space in a separate surgical procedure.

27. The method of claim 24 wherein the membrane covers the portion of the elongate body that may be converted from a generally flexible state to a generally rigid state.

28. The method of claim 24 wherein the membrane covers at least a portion of the elongate body and covers the at least one gripper.

29. The method of claim 22 further comprising actuating a second expandable gripper, wherein the membrane covers both of the grippers and a portion of the bone fixation device located between the grippers.

30. The method of claim 22 wherein the membrane comprises a porous material.

31. The method of claim 22 wherein the membrane comprises a non-porous, generally impervious material.

32. The method of claim 22 wherein the membrane comprises biologically active matter.

33. The method of claim 22 wherein the membrane comprises matter which provides a therapeutic effect to a portion of the body.

34. The method of claim 22 wherein the membrane has a thickness of between 0.010 and 0.030 inches.

35. The method of claim 22 wherein the gripper comprises at least one arm having a distal end that moves outwardly to engage the bone when the gripper is in the expanded configuration.

36. The method of claim 35 wherein the distal end comprises a sharp point.

37. The method of claim 35 wherein the distal end comprises a blunt point.

38. The method of claim 22 wherein the gripper comprises at least two arms that pivot in a scissors fashion relative to each other when the gripper is deployed.

39. The method of claim 22 wherein the membrane has a first portion and a second portion, and wherein the first portion has a thickness that is different than a thickness of the second portion before the gripper is deployed.

40. The method of claim 22 wherein the covering step comprises overmolding the membrane on at least a portion of the bone fixation device.

41. The method of claim 22 wherein the membrane comprises a retainer element molded therein.

42. The method of claim 22 wherein the membrane is elongate and comprises two ends, and wherein the membrane comprises an opening at each of the ends.
